(19) United States
(12) Patent Application Publication
(10) Pub. No.: US 2025/0342971 A1
(43) Pub. Date: Nov. 6, 2025

(54) TECHNIQUES FOR DETECTING MINIMUM RESIDUAL DISEASE

(71) Applicants: Laboratory Corporation of America Holdings, Burlington, NC (US); UCL Business Ltd, London (GB); The Francis Crick Institute Limited, London (GB)

(72) Inventors: Laura Anne Johnson, San Francisco, CA (US); Morgan Schroeder, Boulder, CO (US); Aaron Timothy Garnett, Boulder, CO (US); Abel Licon, Longmont, CO (US); Thomas Dana Harrison, Loveland, CO (US); Christopher Abbosh, London (GB); Charles Swanton, London (GB); Kevin Richard Litchfield, Manchester (GB); Clare Puttick, London (GB)

(73) Assignees: Laboratory Corporation of America Holdings, Burlington, NC (US); UCL Business Ltd, London (GB); The Francis Crick Institute Limited, London (GB)

(21) Appl. No.: 18/871,737

(22) PCT Filed: Jun. 6, 2023
(86) PCT No.: PCT/EP2023/065074

§ 371 Date: Dec. 4, 2024

(30) Foreign Application Priority Data
Jun. 6, 2022 (GB) 2208273.9

(51) Int. Cl. G16H 50/30; G16B 20/20; G16B 30/10; G16H 10/40

(52) U.S. Cl. CPC G16H 50/30; G16B 20/20; G16B 30/10; G16H 10/40

(57) ABSTRACT

The present disclosure describes techniques for determining an indication of minimum residual disease (MRD) in a subject. The indication of MRD may be determined based on sequencing data from a biological sample of the subject. These techniques are performed in part by determining sequencing error and an indication MRD from the same biological sample using the same set of sequencing data.

### RELATED PATENT APPLICATIONS

This patent application claims the benefit of United Kingdom (GB) Patent Application No: 2208273.9 filed on Jun. 6, 2022, entitled “Techniques For Detecting Minimum Residual Disease”, and designated by Mewburn Ellis. The entire content of the foregoing patent application is incorporated herein by reference, including all text, tables and drawings.

### BACKGROUND

A central challenge in treating cancer is early detection of minimum residual disease following cancer treatment. Minimum residual disease (MRD) may be an indicator of cancer recurrence that generally occurs before standard surveillance imaging. One strategy for identifying MRD is monitoring biological samples from the patient for circulating tumor DNA (ctDNA), which can be shed by cancers.

### SUMMARY

Some embodiments of the disclosure provide for a method for determining whether sequencing data of a biological sample of a subject provides an indication that the subject has minimum residual disease. The method comprises using at least one computer hardware processor to perform: (A) obtaining the sequencing data, the sequencing data being previously generated by sequencing the biological sample of the subject, the sequencing data comprising sequence reads covering positions being monitored for mutations; (B) determining, using at least a first subset of the sequence reads, a first value indicative of an expected number of mutations present in the sequencing data due to sequencing error, the determining comprising: determining, using the first subset of sequence reads, a plurality of trinucleotide context (TNC) error rates for a respective plurality of TNC error types; grouping at least some of the plurality of TNC error rates into a plurality of TNC error rate groups; determining TNC group error rates for the plurality of TNC error rate groups using the TNC error rates for the at least some of the plurality of TNC error rates; and determining the first value indicative of the expected number of mutations present in the sequencing data using the TNC group error rates; (C) determining, using at least a second subset of the sequence reads, a second value indicative of an actual number of mutations present at the positions being monitored for mutations; and (D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

In some embodiments, the sequence reads cover at least 10 positions being monitored for mutations. In some embodiments, the sequence reads cover 10-200 positions being monitored for mutations. In some embodiments, the sequence reads cover 50-200 positions being monitored for mutations.

In some embodiments, the method further comprises: obtaining the sequencing data by sequencing the biological sample. In some embodiments, the method further comprises: obtaining the biological sample from a bodily fluid of the subject. In some embodiments, the biological sample comprises circulating tumor DNA (ctDNA). In some embodiments, each of the sequence reads covers at least one of the positions being monitored for mutations. In some embodiments, the sequence reads were obtained using whole exome sequencing. In some embodiments, the sequence reads were obtained using a targeted gene sequencing panel. In some embodiments, the targeted gene sequencing panel targets sequences covering positions being monitored for mutations. In some embodiments, primers are used to amplify the sequences covering positions being monitored for mutations. In some embodiments, the sequences targeted by the targeted gene sequencing panel were determined using sequence data from a primary tumor of the subject.

In some embodiments, the first subset of the sequence reads and the second subset of the sequence reads are the same. In some embodiments, (B) is performed using at least the first subset of the sequence reads and one or more sequence reads in the sequencing data that do not cover the positions being monitored for mutations. In some embodiments, performing (B) further comprises: generating consensus sequence reads using at least the first subset of the sequence reads, wherein each of the consensus sequence reads is generated from those sequence reads, in at least the first subset of the sequence reads, that are associated with a respective common unique molecular identifier (UMI), wherein determining the plurality of trinucleotide context (TNC) error rates for the respective plurality of TNC error types is performed using the generated consensus sequence reads.

In some embodiments, each of the consensus sequence reads is generated from at least a threshold number of sequence reads that are associated with a respective common UMI. In some embodiments, the threshold number of sequence reads is between 2 and 20.

In some embodiments, the method further comprises: selecting a subset of the consensus sequence reads, wherein determining the plurality of trinucleotide context (TNC) error rates for the respective plurality of TNC error types is performed using only the selected subset of consensus sequence reads. In some embodiments, the consensus sequence reads comprise plus strand consensus sequence reads and minus strand consensus sequence reads, and wherein selecting the subset is performed using a criterion that applies a measure of similarity between corresponding plus strand consensus sequence reads and minus strand consensus sequence reads. In some embodiments, the consensus sequence reads comprise plus strand consensus sequence reads and minus strand consensus sequence reads, and selecting a subset of the consensus reads using one or more criteria that apply to the plus strand consensus sequence reads and minus strand consensus sequence reads.

In some embodiments, the method further comprises: determining the measure of similarity between corresponding plus strand consensus sequence reads and minus strand consensus sequence reads. In some embodiments, the consensus sequence reads comprise plus strand consensus sequence reads and minus strand consensus sequence reads, and wherein selecting the subset is performed using a criterion that applies to relative numbers of plus strand consensus sequence reads and corresponding minus strand consensus sequence reads.

In some embodiments, determining the plurality of trinucleotide context (TNC) error rates for the respective plurality of TNC error types using the consensus sequence reads comprises: determining the plurality of TNC error rates using background regions of the consensus sequence reads, wherein the positions being monitored for mutations include a first position, wherein the consensus sequence reads include a first consensus sequence read that covers the first position and the background regions include a first background region for the first consensus sequence read, wherein the first background region comprises nucleotides in the first consensus sequence read that are at least a first threshold distance away from the first position. In some embodiments, the background regions do not include the positions being monitored for mutations

In some embodiments, the consensus sequence reads comprise, for a first position of the positions being monitored for mutations, a first group of plus strand consensus sequence reads associated with a plus strand primer binding sequence beginning at 3′ terminal of each of the plus strand primers in the first group and a second group of minus strand consensus sequence reads associated with a minus strand primer binding sequence beginning at 3′ terminal of the minus strand primer in the second group, wherein determining the plurality of trinucleotide context (TNC) error rates for the respective plurality of TNC error types using the consensus sequence reads comprises: determining the plurality of TNC error rates using: nucleotides, in any sequence read in the first group of plus strand consensus sequence reads, which are located within a second threshold distance of the plus strand primer binding sequence, and nucleotides, in any sequence read in the second group of minus strand consensus sequence reads, which are located within a third threshold distance of the minus strand primer binding sequence. In some embodiments, determining the plurality of trinucleotide context (TNC) error rates using the consensus sequence reads comprises determining a frequency of occurrence of each of the TNC error types in the consensus sequence reads.

In some embodiments, determining the plurality of trinucleotide context (TNC) error rates for the respective plurality of TNC error types using the consensus sequence reads comprises: determining the plurality of TNC error rates from background regions of the consensus sequence reads, wherein the consensus sequence reads include a first consensus sequence read and the background regions include a first background region for the first consensus sequence read, wherein the TNC error rates are determined based on how often each of the TNC error types occurs in the first background region for the first consensus sequence. In some embodiments, the TNC error types correspond to a mutation in any position of a given TNC.

In some embodiments, each of the TNC error types corresponds to a specific mutation of a middle nucleotide in a given TNC.

In some embodiments, the method further comprises: after determining the plurality of trinucleotide context (TNC) error rates and before grouping at least some of the plurality of TNC error rates into a plurality of TNC error rate groups, determining confidence intervals for the TNC error rates; and selecting the at least some of the plurality of TNC error rate for grouping based the confidence intervals for the TNC error rates. In some embodiments, grouping at least some of the plurality of TNC error rates into a plurality of TNC error rate groups comprises clustering the plurality of TNC error rates. In some embodiments, grouping at least some of the plurality of TNC error rates into a plurality of TNC error rate groups comprises grouping using partition around medoids (PAM) clustering. In some embodiments, grouping at least some of the plurality of TNC error rates comprises grouping into 4 TNC error rate groups.

In some embodiments, determining the first value indicative of the expected number of mutations present in the sequencing data is performed using at least some of the TNC group error rates and the number of times at least some of the positions being monitored for mutations are covered by a sequence read in the first subset of sequence reads. In some embodiments, determining the first value indicative of the expected number of mutations present in the sequencing data comprises: determining the first value as a weighted linear combination of the TNC error group rates with each particular one of the TNC error group rates being weighted by a number of times a position being monitored is covered by a sequence read, in the first subset of sequence reads, corresponding to a TNC error type that belongs to that particular TNC error group.

In some embodiments, performing (C) further comprises: generating second consensus sequence reads using at least the second subset of the sequence reads, wherein each of the second consensus sequence reads is generated from those sequence reads, in at least the second subset of the sequence reads, which are associated with a respective common unique molecular identifier (UMI), wherein determining the second value indicative of an actual number of mutations present at the positions being monitored for mutations is performed using the second consensus sequence reads.

In some embodiments, (D) is performed using a statistical hypothesis test having a null hypothesis, by comparing the second value to a distribution associated with the null hypothesis, wherein the distribution has one or more parameters that depend on the first value. In some embodiments, the distribution is a Poisson distribution having a mean value (2) that is set to the first value. In some embodiments, using the statistical hypothesis test comprises determining a measure of likelihood, under the null hypothesis, of observing the actual number of mutations indicated by the second value. In some embodiments, (D) is performed using a one-sided Poisson hypothesis test. In some embodiments, using the one-sided Poisson hypothesis test comprises: setting a mean value (2) of a Poisson distribution to the first value and determining a measure of likelihood, under the Poisson distribution, of observing the actual number of mutations indicated by the second value.

In some embodiments, determining whether the sequencing data provides the indication that the subject has minimum residual disease uses the measure of likelihood. In some embodiments, the subject is likely to have minimum residual disease if the second value indicates that the null hypothesis can be rejected. In some embodiments, (D) further comprises: providing the indication that the subject has minimum residual disease.

In some embodiments, the method further comprises using the at least one computer hardware processor to perform: obtaining one or more of further sequencing data previously generated by sequencing one or more further biological sample(s) of the subject, each of the one or more of further sequencing data comprising further sequence reads covering the positions being monitored for mutations, and for each of the further sequence reads of the one or more of further sequencing data: determining, using at least a first subset of the further sequence reads, a further first value indicative of an expected number of mutations present in a respective sequencing data due to sequencing error, the determining comprising: determining a further plurality of trinucleotide context (TNC) error rates for a respective plurality of TNC error types; grouping at least some of the further plurality of TNC error rates into a further plurality of TNC error rate groups; determining further TNC group error rates for the further plurality of TNC error rate groups using the further TNC error rates for the at least some of the further plurality of TNC error rates; and determining the further first value indicative of the expected number of mutations present in the respective sequencing data using the further TNC group error rates; determining, using at least a second subset of the further sequence reads, a further second value indicative of an actual number of mutations present at the positions; and determining whether the respective sequencing data provides the indication that the subject has minimum residual disease using the further first value indicative of the expected number of mutations present in the respective sequencing data due to sequencing error and the further second value indicative of the actual number of mutations present in the respective sequencing data at the positions being monitored for mutations.

Various aspects described above may be used alternatively or additionally with aspects in any of the systems, methods, and/or processes described herein. Further, a system may be configured to operate according to a method with one or more of the foregoing aspects. Such a system may comprise at least one computer hardware processor, and at least one non-transitory computer-readable medium storing processor executable instructions that, when executed by the at least one computer hardware processor, cause the at least one computer hardware processor to perform such a method. Further, a non-transitory computer-readable medium may comprise processor executable instructions, that when executed by at least one computer hardware processor of a data processing system, cause the at least one computer hardware processor to perform a method with one or more of the foregoing aspects. As such, the foregoing is a non-limiting summary of the invention, which is defined by the attached claims.

### BRIEF DESCRIPTION OF THE DRAWINGS

FIG. 1 is a diagram depicting an illustrative technique 100 for determining an indication of MRD in a subject using sequencing data from a biological sample of the subject and a statistical analysis, according to some embodiments of the technology described herein.

FIG. 2A is a flowchart of an illustrative process 200 for determining an indication of MRD in a subject using sequencing data from a biological sample, according to some embodiments of the technology described herein.

FIG. 2B is a flowchart of an illustrative process 210 for determining an expected number of mutations due to sequencing error, according to some embodiments of the technology described herein.

FIG. 3 illustrates determining a plurality of TNC error rates using the background regions of consensus sequence reads, according to some embodiments of the technology described herein.

FIG. 4 illustrates selecting TNC error types based on their respective error rates, according to some embodiments of the technology described herein.

FIG. 5 illustrates determining TNC group error rates for a respective plurality of TNC error rate groups, according to some embodiments of the technology described herein.

FIG. 6 illustrates aspects of using a statistical test to determine a likelihood that MRD is present using the first value and the second value, according to some embodiments of the technology described herein.

FIG. 7 illustrates aspects of determining an indication of MRD, according to some embodiments of the technology described herein.

FIG. 8 shows boxplots demonstrating the median error-rate (%, y axis) per each of 96 trinucleotide contexts (x axis) transition events from patient primary non-small cell lung cancer (NSCLC) tumor sequencing data.

FIG. 9 shows boxplots demonstrating the median error-rate (%, y axis) per each of 96 trinucleotide contexts (x axis) transversion events from patient primary non-small cell lung cancer (NSCLC) tumor sequencing data.

FIG. 10A shows false positive detection of MRD in patients that did not have clinical cancer relapse (top panel), when the MRD analysis is performed according to some embodiments of the technology described herein.

FIG. 10B shows false positive detection of MRD in patients that had a second primary tumor (bottom panel), which is not expected to be detected by MRD, when the MRD analysis is performed according to some embodiments of the technology described herein.

FIG. 10C shows that MRD is detected on median 151 days before clinical cancer relapse with 37 of 41 cases detected by MRD before clinical relapse, when MRD analysis is performed according to some embodiments of the technology described herein. FIG. 10C also shows that MRD detection is poor in “non-shedding” NSLC with a median lead time on detection 22 days, when MRD analysis is performed according to some embodiments of the technology described herein.

FIG. 10D shows the overlap between the indication of MRD based on sequencing of ctDNA and determining MRD using standard surveillance imaging.

FIG. 11A shows that MRD is generally not detected in non-recurrent patients, when MRD analysis is performed using some embodiments of the technology described herein.

FIG. 11B shows that MRD is almost always detected in post-operative recurrent patients, when MRD analysis is performed using some embodiments of the technology described herein.

FIG. 11C shows that MRD is almost always detected in pre-operative patients, when MRD analysis is performed using some embodiments of the technology described herein.

FIG. 11D simulated MRD negative samples show that at a P-Value <0.1 threshold 121/3157 simulated mock panels were ctDNA positive (in-silico specificity of 96.2%) at a P-Value <0.01 22/3157 simulated mock panels were ctDNA positive (in-silico specificity of 99.3%), when MRD analysis is performed using some embodiments of the technology described herein.

FIG. 11E shows the sensitivity of the MRD analysis (p-value) compared to the amount of spiked in ctDNA into a control sample of background fragmented DNA, when MRD analysis is performed using some embodiments of the technology described herein.

FIG. 12 depicts an illustrative implementation of a computer system that may be used in connection with some embodiments of the technology described herein.

### DETAILED DESCRIPTION

Early detection of cancer relapse/recurrence is an important aspect of effective cancer treatment. One strategy for detecting cancer relapse is searching for minimum residual disease (MRD) in biological samples collected from a subject post cancer therapy. MRD can be determined by sequencing biological samples from a subject to identify circulating tumor DNA (ctDNA), which is indicative of cancer relapse. ctDNA largely contains the same sequence as the wildtype DNA of the subject, save for cancer-associated mutations.

Determining the presence of cancer-associated mutations in cfDNA or ctDNA (an indication of MRD) is challenging for many reasons including sequencing error in sequencing experiments. Sequencing errors may be introduced at multiple points throughout the process of obtaining a sample, preparing the sample, sequencing, and performing post-sequencing analysis of the data. Sequencing error can result in, for example, a false positive identification of MRD because of the false positive appearance of cancer-associated mutations. Thus, methods are needed to correct for false positive appearance of cancer-associated mutations when identifying MRD.

Conventional methods correct for sequencing error by sequencing negative control samples from healthy subjects (see e.g., Abbosh, Christopher, et al., Nature 545.7655 (2017): 446-451.). These methods assume that the sequencing error associated with the healthy subjects and the sequencing error associated with cancer subjects are approximately the same, so a positive MRD call is made when the MRD signal in the cancer subject significantly exceeds the MRD signal in the healthy subject. However, the assumption that sequencing errors are similar between healthy subjects and cancer subjects is not accurate in all cases. Sequencing error between sequencing data collected from different biological samples may be dependent on biological sample collection, biological sample preparation for sequencing, sequencing instrumentation (both type of instrumentation and maintenance of instrumentation), and analysis of sequencing data. As a result, conventional techniques that determine how much sequencing error is present in sequencing data for one sample based on errors found in sequencing data for other samples may lead to inaccurate estimates of error. For example, a recent experiment compared genomic sequencing data from the same cell lines sequenced on different occasions and found false negatives (no mutation when a mutation was expected) 42-51% of the time and false positives (mutation present when no mutation was expected) 5-8% of the time (see Kim, Young-Ho, et al., PloS one 14.9 (2019): e0222535). In contrast to conventional methods, the inventors have developed techniques for determining the amount of sequencing error present in the sample using sequencing data from the very same sample (e.g., sequencing error and an indication of MRD can be determined using the same sequencing data from the same biological sample).

This technique involves determining a sequencing error rate (e.g., a value representing the rate of an incorrect nucleotide being identified at a position; incorrect nucleotides may be identified at a position due to events that take place during sample collection, preparation, sequencing, post-sequence analysis or any other occasion in which the sample or data is manipulated) by monitoring error rates in nucleotides or groups of nucleotides (i.e. nucleotide context (NC)). A nucleotide context (NC) refers to a series of sequential nucleic acids with specific bases in a nucleic acid sequence or a sequence read. In some embodiments error rates in single nucleotides (single nucleotide context) are monitored. In some embodiments error rates in groups of two nucleotides (di-nucleotide context), three nucleotides (trinucleotide context), four nucleotides (four nucleotide context), five nucleotides (five nucleotide context), six nucleotides (six nucleotide context) or more are monitored. In some embodiments, error rates in groups of trinucleotide context are monitored as described herein. In turn, the estimated sequencing error rate may be compared to the actual number of mutations observed in the positions being monitored for mutations to determine an indication of MRD. In some embodiments, this technique involves estimating sequencing error from sequencing results at positions not being monitored for cancer-associated mutations (the collection of such sequence read positions may be termed “background regions” herein).

Accordingly, some embodiments provide for a computer-implemented method for determining whether sequencing data of a biological sample (e.g., plasma) of a subject (e.g., a human subject) provides an indication that the subject has minimum residual disease. In some embodiments, the method comprises: (A) obtaining the sequencing data, the sequencing data being previously generated by sequencing the biological sample of the subject, the sequencing data comprising sequence reads covering positions being monitored for mutations (e.g., the positions may be determined by analyzing results of sequencing of a primary tumor of the subject to identify positions informative for subsequent monitoring for MRD); (B) determining, using at least a first subset of the sequence reads (e.g., first subset that may be selected based on data quality), a first value indicative of an expected number of mutations present in the sequencing data due to sequencing error, the determining comprising: determining, using the first subset of sequence reads, a plurality of nucleotide context (NC) error rates selected from single nucleotide context, dinucleotide context, trinucleotide context, four nucleotide context, five nucleotide context, and six nucleotide context (e.g., rate at which a nucleotide repeat may be mutated due to sequencing error) for a respective plurality of NC error types (e.g., different types of mutations that may be observed in a nucleotide context); grouping at least some of the plurality of NC error rates into a plurality of NC error rate groups (e.g., using PAM-clustering, k-nearest neighbors clustering, or hierarchical agglomerative clustering; grouping may decrease statistical noise by increasing the number of sequence reads used to determine NC group error rate, which is in turn used to determine the first value); determining NC group error rates for the plurality of NC error rate groups using the NC error rates for the at least some of the plurality of NC error rates (e.g., population weighted average of NC error rates may be a NC group error rate); and determining the first value indicative of the expected number of mutations present in the sequencing data using the NC group error rates; (C) determining, using at least a second subset of the sequence reads, a second value indicative of an actual number of mutations present at the positions being monitored for mutations (e.g., the number of times mutations are present at the positions being monitored for mutations); and (D) determining whether the sequencing data provides the indication that the subject has minimum residual disease (e.g., the probability that the subject has MRD) using the first value indicative of the expected number of mutations present in the sequencing data due to sequencing error and the second value indicative of the actual number of mutations present in the sequencing data at the positions being monitored for mutations (e.g., using a one-sided Poisson test where the first value is A).

Although embodiments, examples, claims and drawings herein often refer to tri-nucleotide context (TNC), it is understood that the methods described in the embodiments, examples, claims and drawings herein can be performed using any suitable nucleotide context (e.g., single nucleotide context (SNC), dinucleotide context (DNC), trinucleotide context (TNC), four-nucleotide context (4NC), five-nucleotide context (5NC), six-nucleotide context (6NC), and the like).

Accordingly, some embodiments provide for a computer-implemented method for determining whether sequencing data of a biological sample (e.g., plasma) of a subject (e.g., a human subject) provides an indication that the subject has minimum residual disease. In some embodiments, the method comprises: (A) obtaining the sequencing data, the sequencing data being previously generated by sequencing the biological sample of the subject, the sequencing data comprising sequence reads covering positions being monitored for mutations (e.g., the positions may be determined by analyzing results of sequencing of a primary tumor of the subject to identify positions informative for subsequent monitoring for MRD); (B) determining, using at least a first subset of the sequence reads (e.g., first subset that may be selected based on data quality), a first value indicative of an expected number of mutations present in the sequencing data due to sequencing error, the determining comprising: determining, using the first subset of sequence reads, a plurality of nucleotide context (NC) error rates (e.g., rate a nucleotide or group of sequential nucleotides in a sequence may be mutated due to sequencing error) for a respective plurality of NC error types (e.g., different types of mutations that may be observed in a NC); grouping at least some of the plurality of NC error rates into a plurality of NC error rate groups (e.g., using PAM-clustering; grouping may decrease statistical noise by increasing the number of sequence reads used to determine NC group error rate, which is in turn used to determine the first value); determining NC group error rates for the plurality of NC error rate groups using the NC error rates for the at least some of the plurality of NC error rates (e.g., population weighted average of NC error rates may be a NC group error rate); and determining the first value indicative of the expected number of mutations present in the sequencing data using the NC group error rates; (C) determining, using at least a second subset of the sequence reads, a second value indicative of an actual number of mutations present at the positions being monitored for mutations (e.g., the number of times mutations are present at the positions being monitored for mutations); and (D) determining whether the sequencing data provides the indication that the subject has minimum residual disease (e.g., a probability or likelihood that the subject has MRD) using the first value indicative of the expected number of mutations present in the sequencing data due to sequencing error and the second value indicative of the actual number of mutations present in the sequencing data at the positions being monitored for mutations (e.g., using a one-sided Poisson test where the first value is lambda).

Accordingly, some embodiments provide for a computer-implemented method for determining whether sequencing data of a biological sample (e.g., plasma) of a subject (e.g., a human subject) provides an indication that the subject has minimum residual disease. In some embodiments, the method comprises: (A) obtaining sequencing data, the sequencing data being previously generated by sequencing the biological sample of the subject, the sequencing data comprising sequence reads covering positions being monitored for mutations (e.g., the positions may be determined by analyzing results of sequencing of a primary tumor of the subject to identify positions informative for subsequent monitoring for MRD); (B) determining, using at least a first subset of the sequence reads (e.g., first subset that may be selected based on data quality), a first value indicative of an expected number of mutations present in the sequencing data due to sequencing error, the determining comprising: determining, using the first subset of sequence reads, a plurality of trinucleotide context (TNC) error rates (e.g., rate a three-nucleotide repeat may be mutated due to sequencing error) for a respective plurality of TNC error types (e.g., different types of mutations that may be observed in a TNC); grouping at least some of the plurality of TNC error rates into a plurality of TNC error rate groups (e.g., using PAM-clustering; grouping may decrease statistical noise by increasing the number of sequence reads used to determine TNC group error rate, which is in turn used to determine the first value); determining TNC group error rates for the plurality of TNC error rate groups using the TNC error rates for the at least some of the plurality of TNC error rates (e.g., population weighted average of TNC error rates may be a TNC group error rate); and determining the first value indicative of the expected number of mutations present in the sequencing data using the TNC group error rates; (C) determining, using at least a second subset of the sequence reads, a second value indicative of an actual number of mutations present at the positions being monitored for mutations (e.g., the number of times mutations are present at the positions being monitored for mutations); and (D) determining whether the sequencing data provides the indication that the subject has minimum residual disease (e.g., a probability or likelihood that the subject has MRD) using the first value indicative of the expected number of mutations present in the sequencing data due to sequencing error and the second value indicative of the actual number of mutations present in the sequencing data at the positions being monitored for mutations (e.g., using a one-sided Poisson test where the first value is lambda).

In some embodiments, coverage and/or resolution play a significant role in determining an optimal context size (e.g., NC) for determining error rates, for example where coverage refers to a maximum number of observations for an error rate context, on average, given a depth of sequencing for the sample, and where resolution refers to a total number of error rate contexts of a given size. Sometimes, a larger context size yields more contexts, following the formula (N=3*4\ddot{ }k) where “k” is the context size, for example. More contexts (i.e. higher resolution) often allows for more accurate estimation of an error rate that is driven by the bias of the sequence surrounding a variant. This sometimes comes at a direct and proportional cost of potential coverage (Depth/N). For example, at an example minimum depth of 10,000 reads for a sample, a trinucleotide context has a theoretical potential to detect error rates down to 1/52 (1.9%) on average while still increasing the overall resolution vs. di-or mono-nucleotide contexts. The inventors herein have found that a tri-nucleotide context is often the largest context size that yields acceptable detectable error rates across many sequencing depths, and therefore often provides for a technical advantage over larger or smaller nucleotide context.

In some embodiments, the biological sample may be plasma and may comprise cell free DNA and/or ctDNA. Aspects of biological samples are described herein including in the section below called “Biological Samples”.

In some embodiments, the subject may be a human subject that has been previously treated for cancer (e.g., lung cancer). Various subjects and cancer types are described herein including in the sections below called “Subjects”.

In some embodiments, an indication of minimum residual disease may be determined using a statistical test (e.g., a statistical hypothesis test, which may be a one-sided or a two-sided test, and may be a Poisson test, for example). Aspects of statistical tests are described herein. In some embodiments, minimum residual disease (MRD) may be an indicator of cancer recurrence that generally occurs before standard surveillance imaging detects cancer recurrence. Aspects of minimum residual disease (MRD) are described herein including in the section below called “Minimum Residual Disease (MRD)”.

In some embodiments, obtaining the sequencing data may include sequencing nucleic acids in the biological sample to obtain sequencing data. Aspects of sequencing data are described herein including in the section below called “Sequencing Data”. In some embodiments, the sequence reads covering sequences monitored for mutations additionally may cover regions that are not being monitored for mutations (e.g., background regions). Thus, sequencing data from a biological sample of a subject may comprise sequence reads that may be used to monitor positions being monitored for mutations and to determine sequencing error. In some embodiments, the positions being monitored for mutations may have been determined previously by sequencing the primary tumor of the subject. Aspects of positions being monitored for mutations are described herein including in the section below called “Positions Being Monitored for Mutations”. In some embodiments, at least 10, 10-200, or 50-200 positions are monitored for mutations. Sequencing data may be obtained using a suitable method. In some embodiments, the sequencing data may be obtained using whole genome sequencing. In some embodiments, the sequencing data may be obtained using whole exome sequencing. In some embodiments, the sequencing data may be obtained using a targeted gene sequencing panel or method. Aspects of the targeted gene sequencing panel are described herein including in the section below called “Sequencing Data”.

As described above, in some embodiments, the method comprises (B) determining, using at least a first subset of the sequence reads, a first value indicative of an expected number of mutations present in the sequencing data due to sequencing error. In some embodiments, the first subset of sequence reads comprises any number or combination of sequence reads in the sequencing data. For example, the first subset may comprise consensus sequence reads. In some embodiments, consensus sequence reads can be identified or determined using a suitable alignment method (e.g., Pileup, Bowtie, BarraCUDA, BFAST, CUSHAW, ELAND, FASTA, SOAP, the like, variations or combinations thereof). Consensus sequence reads may be determined using a plurality of sequence reads having the same unique molecular identifier (e.g., barcode). In particular, the first subset may comprise deep consensus sequence reads. In some embodiments, a deep consensus read may be a consensus read that may be determined using at least 2 sequences reads, at least 3 sequence reads, at least 4 sequence reads, at least 5 sequence reads, at least 6 sequence reads, at least 7 sequence reads, at least 8 sequence reads, at least 9 sequence reads, at least 10 sequence reads, at least 15 sequence reads, or at least 20 sequence reads having the same unique molecular identifier.

In some embodiments, the first value may be determined using sequence reads covering one or more positions being monitored for a plurality of mutations (e.g., cancer associated mutations). However, in other embodiments, the first value may be determined using one or more sequence reads each covering one or more positions being monitored for a plurality of mutations (e.g., cancer-associated mutations) and one or more sequence reads each not covering any positions being monitored for a plurality of mutations. Yet in other embodiments, the first value may be determined using only sequence reads that do not cover any position being monitored for cancer-associated mutations.

In some embodiments, performing (B) comprises generating consensus sequence reads using at least the first subset of the sequence reads, wherein each of the consensus sequence reads may be generated from a plurality of those sequence reads, in at least the first subset of the sequence reads, the plurality of those sequence reads associated with a respective common unique molecular identifier (UMI). In some embodiments, generating consensus reads using UMIs may mitigate polymerase chain reaction amplification bias, which may occur during sample preparation for sequencing. Aspects of consensus sequence reads are described herein. In some embodiments, each of the consensus sequence reads may be generated from at least a threshold number of sequence reads that are associated with a respective common UMI (e.g., 2-20). In some embodiments, the method comprises selecting a subset of consensus sequence reads, wherein determining the plurality of NC error rates (e.g., trinucleotide context (TNC) error rates) for the respective plurality of NC or TNC error types is performed using only the selected subset of consensus sequence reads. In some embodiments, the consensus sequence reads comprise plus strand consensus sequence reads and minus strand consensus sequence reads, and wherein selecting the subset may be performed based on a measure of similarity between corresponding plus strand consensus sequence reads and minus strand consensus sequence reads. In some embodiments, the consensus sequence reads comprise plus strand consensus sequence reads and minus strand consensus sequence reads, and selecting a subset of the consensus reads using one or more criteria that apply to the plus strand consensus sequence reads and minus strand consensus sequence reads. In some embodiments, the method comprises determining the measure of similarity between corresponding plus strand consensus sequence reads and minus strand consensus sequence reads using a suitable statistical test. In some embodiments, the consensus sequence reads comprise plus strand consensus sequence reads and minus strand consensus sequence reads, and wherein selecting the subset may be performed based on relative numbers of plus strand consensus sequence reads and corresponding minus strand consensus sequence reads. Thus, by including a threshold and making these selections, in some embodiments, consensus reads may be more likely to be representative of the actual DNA sequences from the subject.

In some embodiments, a nucleotide context (NC) refers to a specific base in a nucleic acid sequence or a sequence read. In some embodiments, a nucleotide context (NC) refers to a series of sequential nucleic acids (e.g., 2, 3, 4, 5, 6, 7, 8 or more sequential nucleic acids) with specific bases in a nucleic acid sequence or a sequence read. A NC error type refers to a specific mutation (with reference to a wildtype and/or reference genome) in any given NC. For example, for any single NC there are three possible error types (e.g., for an A nucleotide, there is A>T, A>C, or A>G). In yet another example, for a dinucleotide context with wild type sequence AT, the NC error types may include, but are not limited to, AA, AG, AC, GT, GA, GC, GG, CT, CA, CC, CG, TT, TA, TC and TG. A NC error rate may refer to a frequency at which each NC error type occurs within a NC. In some embodiments, only a select NC error type, or select plurality of NC error types may be considered.

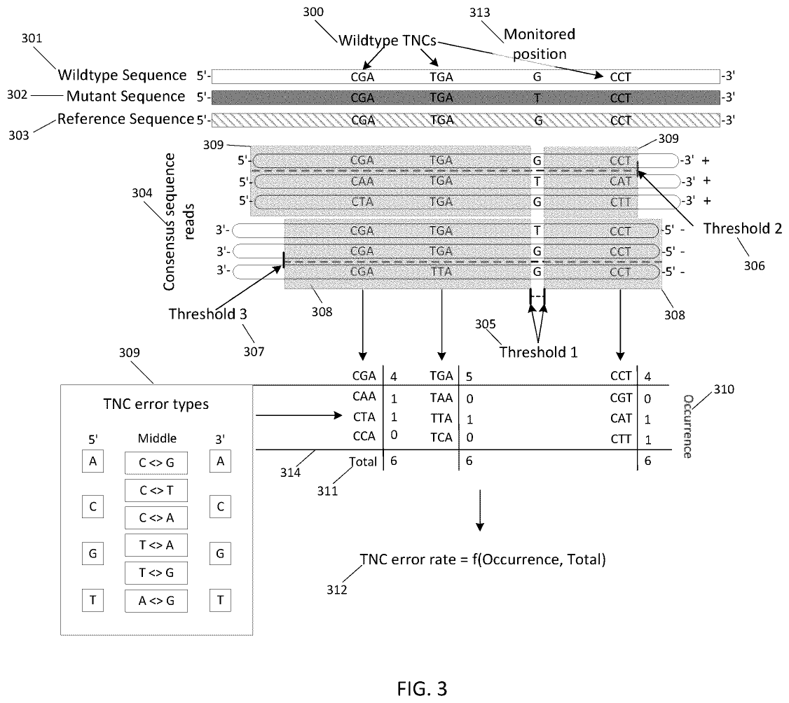

A trinucleotide context (TNC) refers to a series of three sequential nucleic acids with specific bases in a nucleic acid sequence or a sequence read (e.g., TAC). Aspects of trinucleotide context are described herein including in the section below called “Trinucleotide Context (TNC)”. A TNC error type refers to a specific mutation (with reference to a wildtype and/or reference genome) in any given TNC (e.g., A>T, A>C, or A>G). For example, if the expected (wildtype) TNC is TAC then the TNC error types may include, but are not limited to, TTC, TCC, AAC, TAG, CAC, GAC, TAT, TAA and TGC. TNC error types are described further herein including in reference to FIG. 3. A TNC error rate may refer to the frequency at which each TNC error type occurs within the context of a TNC. In some embodiments, only a respective plurality of TNC error types may be considered. For example, in some embodiments, the respective plurality of TNC error types may refer to TNC error types where the middle position of the TNC is mutated and the first (3′) and third (5′) positions may not be mutated.

In some embodiments, determining the plurality of trinucleotide context (TNC) error rates for the respective plurality of TNC error types using sequence reads (e.g., consensus sequence reads) comprises: determining the plurality of TNC error rates using background regions of the consensus sequence reads, wherein the positions being monitored for mutations include a first position, wherein the consensus sequence reads include a first consensus sequence read that covers the first position and the background regions include a first background region for the first consensus sequence read, wherein the first background region comprises nucleotides in the first consensus sequence read that are at least a first threshold distance away from the first position. Thus, TNC error rates may be determined using TNCs that are a threshold distance away from the position being monitored for mutations. In some embodiments, the threshold distance is used to exclude TNCs that have high error (e.g., it is known that nucleotides at the end of a sequence read may be lower confidence than those at the beginning of a sequence read). Aspects of TNC error rates and background regions are described herein and, in the section below with reference to FIG. 3. In some embodiments, the background regions do not include the positions being monitored for mutations.

In some embodiments, the consensus sequence reads comprise, for a first position of the positions being monitored for mutations, a first group of plus strand consensus sequence reads associated with a plus strand primer binding sequence at 3′ terminal of each of the plus strand consensus sequence reads in the first group and a second group of minus strand consensus sequence reads associated with a minus strand primer binding sequence at 3′ terminal of the minus strand consensus sequence reads in the second group, wherein determining the plurality of trinucleotide context (TNC) error rates for the respective plurality of TNC error types using the consensus sequence reads comprises: determining the plurality of TNC error rates using: nucleotides, in any sequence read in the first group of plus strand consensus sequence reads, which are located within a second threshold distance of the plus strand primer binding sequence, and nucleotides, in any sequence read in the second group of minus strand consensus sequence reads, which are located within a third threshold distance of the minus strand primer binding sequence. Thus, TNC error rates may be determined using TNCs that are a threshold distance away from the beginning of a sequence read as determined by the location of the plus strand primer binding sequence or minus strand primer binding sequence. Aspects of the second threshold and third threshold are described herein including in the section below with reference to FIG. 3.

In some embodiments, determining the plurality of trinucleotide context (TNC) error rates for the respective plurality of TNC error types using the consensus sequence reads comprises: determining the plurality of TNC error rates from background regions of the consensus sequence reads, wherein the consensus sequence reads include a first consensus sequence read and the background regions include a first background region for the first consensus sequence read, wherein the TNC error rates are determined based on how often each of the TNC error types occurs in the first background region for the first consensus sequence. Thus, TNC error rates, in some embodiments, may be calculated using only background regions of sequence reads. In other embodiments, TNC error rate may be calculated using both background regions and positions being monitored for mutations; such embodiments rely upon the understanding that vast majority of TNCs in the sequence reads are not being monitored for mutations, so the positions that are being monitored for mutations may be of such a small number that including them when determining sequencing error, in some embodiments, may not substantively change the sequencing error.

In some embodiments, the method comprises identifying and/or removing positions in background regions with an error rate of >=0.5%, >=1%, >=1.5%, >=2%, or >=3%. This may remove positions that could bias estimation of background error (e.g. positions where a genomic sequence of a biological sample truly differs from a reference genomic sequence). In some embodiments, a reference genomic sequence is a patient-specific genomic sequence (e.g., wild-type sequence). In some embodiments, a reference genomic sequence is a reference genome (e.g., hg19).

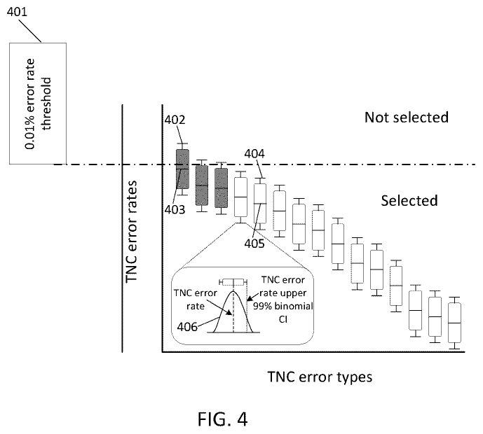

In some embodiments, the method further comprises: after determining the plurality of trinucleotide context (TNC) error rates and before grouping at least some of the plurality of TNC error rates into a plurality of TNC error rate groups, determining confidence intervals for the TNC error rates (e.g., a 99% binomial confidence interval for each TNC error rate); and selecting the at least some of the plurality of TNC error rates for grouping based on the confidence intervals for the TNC error rates when the confidence intervals exceed a threshold (e.g., a 99% binomial confidence interval based on of all of the TNC error rates. This step may allow the highest TNC error rates (which may be anomalously high and decrease algorithm accuracy) to be removed prior to determining the first value. Aspects of selecting TNC error rates for grouping are discussed herein including with reference to FIG. 4. Thus, in some embodiments, high TNC error rates may be removed prior to determining the first value.

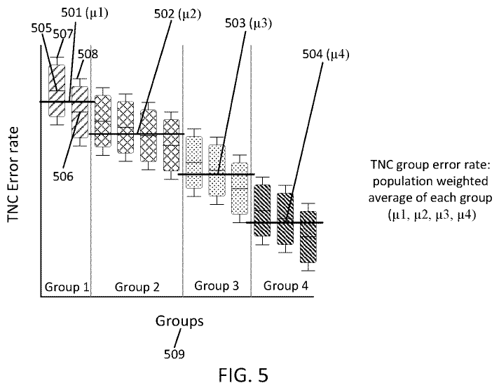

Aspects of grouping TNC error rates are described herein including with reference to FIG. 5. In some embodiments, each TNC error rate group comprises at least 1 TNC error rate. In some embodiments, grouping at least some of the plurality of TNC error rates into a plurality of TNC error rate groups comprises grouping using a suitable clustering method, non-limiting examples of which include k-means clustering, hierarchical agglomerative clustering, partition around medoids (PAM) clustering, and the like. In some embodiments, grouping at least some of the plurality of TNC error rates into a plurality of TNC error rate groups comprises grouping using partition around medoids (PAM) clustering. In some embodiments, grouping at least some of the plurality of TNC error rates comprises grouping into 4 TNC error rate groups. Grouping TNC error rates into TNC error rate groups may be performed, in some embodiments, in order to have sufficient sequence read depth for determination of TNC group error rate.

In some embodiments, determining the first value indicative of the expected number of mutations present in the sequencing data may be performed using at least some of the TNC group error rates and using the number of times at least some of the positions being monitored for mutations are covered by a sequence read in the first subset of sequence reads. In some embodiments, the TNC group error rate for a given TNC error rate group may be determined by calculating the population weighted average of the TNC error rate group. In some embodiments, the number of times at least some of the positions being monitored for mutations are covered by a sequence read in the first subset of sequence reads may be determined by counting the number of times each position being monitored is covered by a sequence read in the subset of sequence reads of the sequencing data. Aspects of TNC group error rate are described herein including with reference to FIG. 5.

In some embodiments, the determining the first value indicative of the expected number of mutations present in the sequencing data comprises: determining the first value as a weighted linear combination of the TNC error group rates with each particular one of the TNC error group rates being weighted by a number of times a position being monitored for mutations is covered by a sequence read in the first subset of sequence reads, corresponding to a TNC error type that belongs to that particular TNC error group. Thus, in some embodiments, the first value may be calculated based on information from all of the TNC error rate groups, which increases the data used to estimate TNC error rates and may provide an improved estimation of TNC error rate.

The methods described herein for determining and using TNC error group rates can be applied to any suitable NC (e.g., single NC, dinucleotide context, TNC, and the like).

As described above, in some embodiments, the method comprises (C) determining, using at least a second subset of the sequence reads, a second value indicative of an actual number of mutations present at the positions being monitored for mutations. In some embodiments, the second subset of sequence reads comprises sequences reads comprising positions being monitored for mutations. In some embodiments, the first subset of the sequence reads and the second subset of the sequence reads may be the same subset of sequence reads. In some embodiments, the second value may be determined based on counting the number of times each position being monitored for mutations is mutated in the second subset of reads. In some embodiments, performing (C) further comprises: generating second consensus sequence reads using at least the second subset of the sequence reads, wherein each of the second consensus sequence reads may be generated from those sequence reads, in at least the second subset of the sequence reads, which are associated with a respective common unique molecular identifier (UMI), wherein determining the second value indicative of an actual number of mutations present at the positions being monitored for mutations may be performed using the second consensus sequence reads. In some embodiments, the second subset of consensus sequence reads may include consensus reads constructed using 2-20 reads. Thus, similar to calculation of the first value, in some embodiments, the second value may also be calculated using consensus reads for the same reasons (e.g., controlling for PCR amplification bias).

As described above, in some embodiments, the method comprises (D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations. In some embodiments, (D) may be performed using a suitable statistical test (e.g., one-sided Poisson test or a t-test) having a null hypothesis, wherein a distribution associated with the null hypothesis has one or more parameters that depend on the first value. In some embodiments, the distribution may be a Poisson distribution having a mean value (2) that may be set to the first value. In some embodiments, using a statistical test comprises determining a measure of likelihood, under the null hypothesis, of observing the actual number of mutations indicated by the second value. In some embodiments, the subject is likely to have minimum residual disease if the second value indicates that the null hypothesis can be rejected. In some embodiments, performing the one-sided Poisson hypothesis test comprises: setting a mean value (2) of a Poisson distribution to the first value and determining a measure of likelihood, under the Poisson distribution, of observing the actual number of mutations indicated by the second value. In some embodiments, the sequencing data may provide an indication that the subject has minimum residual disease (MRD) using the measure of likelihood from the Poisson test. In some embodiments, an indication of MRD may be based on a p-value from the statistical test that is below a pre-determined alpha (e.g., p-value≤0.01). In some embodiments, rejection of the null hypothesis of the Poisson test may be an indication of MRD. In some embodiments, failure to reject the null hypothesis of a Poisson test may not be an indication of MRD. In some embodiments, (D) further comprises: providing the indication that the subject has minimum residual disease. Aspects of the indication of MRD are described herein including in the section below called “Indication of Minimum Residual MRD”.

In some embodiments, a presence of MRD is determined (e.g., as in (D)) using a Chi-squared test across each monitored position comparing Observed vs. Expected. In some embodiments, positions with zero deep alternate observations (DAOs) are filtered out.

In some embodiments, a presence of MRD is determined (e.g., as in (D)) by subtracting the first value indicative of the expected number of mutations present in the sequencing data from the second value indicative of an actual number of mutations present at the positions being monitored for mutations, and determining if the difference is greater than a threshold value. In some embodiments a threshold value can be zero.

In some embodiments, any of the embodiments of this method may be repeated as described above using an additional biological sample collected from the subject. In some embodiments, the methods described herein may encompass a method of monitoring for MRD comprising obtaining one or more further sequencing data previously generated by sequencing one or more further biological sample(s) from the subject over time and analyzing these samples accordingly to the embodiments described above. Thus, in some embodiments, a subject may be incrementally monitored for MRD over a period of weeks, months, years, or throughout the remainder of the subject's lifespan. In some embodiments, the method further comprises, upon identification of MRD, referring the subject for treatment and upon not identifying MRD not referring the subject for treatment.

In some embodiments, the method comprises: obtaining second sequencing data previously generated by sequencing a second biological sample (e.g., additional biological sample) of the subject, the second sequencing data comprising second sequence reads covering the positions being monitored for mutations; determining, using at least a first subset of the second sequence reads, a third value indicative of an expected number of mutations present in the second sequencing data due to sequencing error (e.g., determined in same way as the first value, but with second sequencing data), the determining comprising: determining a second plurality of trinucleotide context (TNC) error rates for the respective plurality of TNC error types; grouping at least some of the second plurality of TNC error rates into a second plurality of TNC error rate groups; determining second TNC group error rates for the second plurality of TNC error rate groups using the second TNC error rates for the at least some of the second plurality of TNC error rates; and determining the third value indicative of the expected number of mutations present in the second sequencing data using the second TNC group error rates (e.g., determined in same way as the first value, but with second sequencing data); determining, using at least a second subset of the second sequence reads, a fourth value indicative of an actual number of mutations present at the positions; and determining whether the second sequencing data provides the indication that the subject has minimum residual disease using the third value indicative of the expected number of mutations present in the second sequencing data due to sequencing error and the fourth value indicative of the actual number of mutations present in the second sequencing data at the positions being monitored for mutations.

In some embodiments, the method further comprises using the at least one computer hardware processor to perform: obtaining one or more of further sequencing data (e.g., a second sequence data, a third sequence data, etc.) previously generated by sequencing one or more further biological sample(s) (e.g., a second biological sample, a third biological sample, etc. collected overt time) of the subject, each of the one or more of further sequencing data comprising further sequence reads covering the positions being monitored for mutations, and for each of the further sequence reads of the one or more of further sequencing data: determining, using at least a first subset of the further sequence reads, a further first value indicative of an expected number of mutations present in a respective sequencing data due to sequencing error, the determining comprising: determining a further plurality of trinucleotide context (TNC) error rates for a respective plurality of TNC error types; grouping at least some of the further plurality of TNC error rates into a further plurality of TNC error rate groups; determining further TNC group error rates for the further plurality of TNC error rate groups using the further TNC error rates for the at least some of the further plurality of TNC error rates; and determining the further first value indicative of the expected number of mutations present in the respective sequencing data using the further TNC group error rates; determining, using at least a second subset of the further sequence reads, a further second value indicative of an actual number of mutations present at the positions; and determining whether the respective sequencing data provides the indication that the subject has minimum residual disease using the further first value indicative of the expected number of mutations present in the respective sequencing data due to sequencing error and the further second value indicative of the actual number of mutations present in the respective sequencing data at the positions being monitored for mutations.

Some embodiments of the disclosure provide for a method for determining whether sequencing data of a biological sample of a subject has a mutation (e.g., a cancer-associated mutation). In these embodiments, the method may comprise using at least one computer hardware processor to perform: (A) obtaining the sequencing data, the sequencing data being previously generated by sequencing the biological sample of the subject, the sequencing data comprising sequence reads covering a position being monitored for mutations; (B) determining, using at least a first subset of the sequence reads, a first value indicative of an expected number of mutations present in the sequencing data due to sequencing error, the determining comprising: determining, using the first subset of sequence reads, a plurality of trinucleotide context (TNC) error rates for a respective plurality of TNC error types; grouping at least some of the plurality of TNC error rates into a plurality of TNC error rate groups; determining TNC group error rates for the plurality of TNC error rate groups using the TNC error rates for the at least some of the plurality of TNC error rates; and determining the first value indicative of the expected number of mutations present in the sequencing data using the TNC group error rates; (C) determining, using at least a second subset of the sequence reads, a second value indicative of an actual number of mutations present at the position being monitored for mutations; and (D) determining whether the sequencing data provides the indication that the subject has a mutation at the positions being monitored for mutations using the first value indicative of the expected number of mutations present in the sequencing data due to sequencing error and the second value indicative of the actual number of mutations present in the sequencing data at the position being monitored for mutations.

Following below are more detailed disclosures of the various concepts and embodiments related to and the method and compositions of indicating MRD described herein.

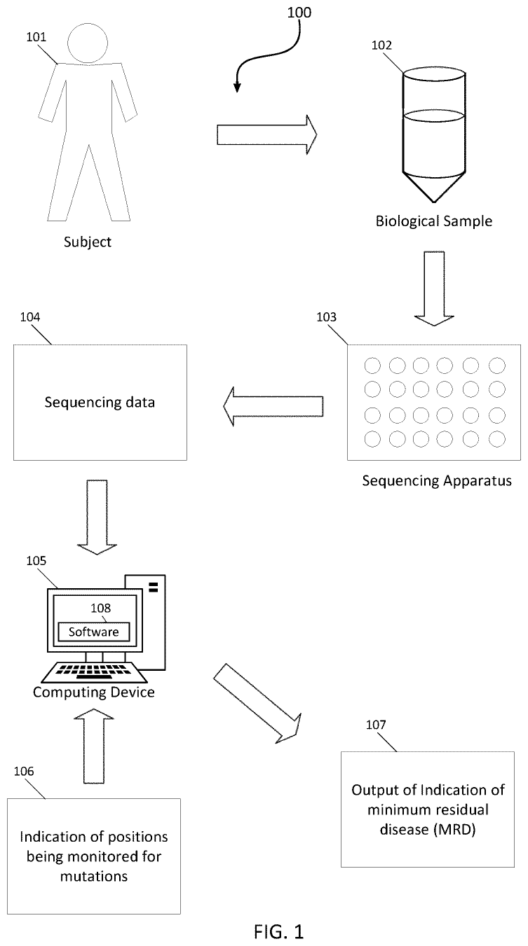

FIG. 1 is a diagram depicting an illustrative technique 100 for determining and outputting indication of MRD 107 in a subject 101. Technique 100 involves collecting a biological sample 102 (e.g., plasma) from a subject 101, sequencing the polynucleotides (e.g., ctDNA) from the biological sample using a sequencing apparatus 103 to generate sequencing data 104, inputting the sequencing data 104 and positions being monitored for mutations 106, into a computing device 105 (e.g., laptop computer, desktop computer, one or more servers, a cloud computing device, a smart phone, tablet, and/or any other suitable computing device) that is configured to execute software 108 that, when executed, causes the computing device 105 to perform the techniques described herein for detecting an indication of MRD.

### Subject

In some embodiments, the subject may be a mammal (e.g., a human, a non-human primate, a dog, a cat, a horse, a goat, a sheep, a mouse, or a rat), a bird, a reptile, an amphibian, a fish, or a laboratory model organism (e.g., mice and rats). In some embodiments, the subject may be a human. In some embodiments, the subject may be an adult human (e.g., older than 18 years of age). In some embodiments, the subject may be a human child. In some embodiments, the subject may be a human infant.

In some embodiments, the subject may be in remission from a disease. In some embodiments, the subject may have been treated for a disease (e.g., cancer). In some embodiments, the subject may have been previously treated using one or more of surgery, chemotherapy, radiation therapy, immunotherapy, and/or hormone therapy. In some embodiments, the subject may be in remission from cancer. In some embodiments, the subject may be in remission from lung cancer, brain cancer, liver cancer, kidney cancer, immune cancer, breast cancer, skin cancer, bone cancer, uterine cancer, prostate cancer, testicular cancer, or colon cancer. In some embodiments, the subject may be in remission of non-small cell lung cancer (NSCLC). In some embodiments, the subject may be in remission of small cell lung cancer (SCLC). In some embodiments, the subject may be in remission of lung adenocarcinoma. In some embodiments, the subject may be in remission of squamous cell carcinoma. In some embodiments, the subject may be in remission of melanoma. In some embodiments, the biological sample comprises ctDNA released by cancer cells into a bodily fluid of the subject. In some embodiments, the subject may be in remission of a cancer selected from NSCLC, colorectal cancer (CRC), bladder cancer, pancreatic cancer, head and neck squamous cell carcinomas (HNSCC), breast cancer, and hematological cancers (e.g., leukemia, lymphoma, and multiple myeloma). These cancers may be particularly likely to release DNA in bodily fluids.

In some embodiments, a subject has, or is suspected of having a cancer or tumor.

### Biological Sample

As shown in FIG. 1, a biological sample 102 is collected from the subject. In some embodiments, a biological sample comprises any cell, tissue, biological fluid, or bone from a subject, or any other portion of a subject. In some embodiments, the biological sample comprises ctDNA of a subject. In some embodiments, the biological sample may be a tissue biopsy (e.g., a tumor biopsy). In some embodiments, the tissue may be a brain tissue, lung tissue, liver tissue, kidney tissue, skin tissue, pancreatic tissue, connective tissue, muscle tissue, or nervous tissue. In some embodiments, the biological sample may be a biological fluid from a subject. In some embodiments, the biological fluid may be saliva, semen, vaginal secretions, urine, feces, nasal mucus, sweat, ear wax, spinal fluid, blood, serum, or plasma from a subject. In some embodiments, the biological sample may be plasma from a subject. In some embodiments, the biological sample from a subject may be blood or a blood product (e.g., serum or plasma), wherein the blood or blood product comprises tumor cells and/or ctDNA. In some embodiments, a biological sample (e.g., a blood product, blood, plasma or serum) comprises cell free DNA (cfDNA) or ctDNA. Cell free DNA derived from a subject having a cancer or tumor often comprises ctDNA.

In some embodiments, the biological sample may be collected from any portion of the subject's body including but not limited to hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, any fluid [such as blood (e.g., whole blood, blood serum, or blood plasma), saliva, tears, synovial fluid, cerebrospinal fluid, pleural fluid, pericardial 5 fluid, ascitic fluid, and/or urine], and/or any type of tissue (e.g., muscle tissue, epithelial tissue, connective tissue, or nervous tissue.

Any of the biological samples described herein may be collected using any suitable technique, including as described in Biospecimens and biorepositories: from afterthought to science by Vaught et al. (Cancer Epidemiol Biomarkers Prev. 2012 February;21 (2): 253-5), and Biological sample collection, processing, storage and information management by Vaught and Henderson (IARC Sci Publ. 2011; (163): 23-42).

In some embodiments, multiple biological samples may be collected from a subject and sequenced to obtain sequencing data. In some embodiments, the multiple biological samples may be sequentially collected from a subject over a specified period of time then sequenced to obtain sequencing data. In some embodiments, the specified period of time may begin after cancer treatment ends and may continue for the remainder of the subject's life. In some embodiments, the frequency with which biological samples are collected from a subject may be any suitable frequency for monitoring MRD. In some embodiments, biological samples may be collected from a subject weekly. In some embodiments, biological samples may be collected from a subject about twice a month. In some embodiments, biological samples may be collected from a subject about once a month. In some embodiments, biological samples may be collected from a subject about once every three months. In some embodiments, biological samples may be collected from a subject about once every six months. In some embodiments, biological samples may be collected from a subject at least twice a month. In some embodiments, biological samples may be collected from a subject at least once a month. In some embodiments, biological samples may be collected from a subject at least once every three months. In some embodiments, biological samples may be collected from a subject at least once every six months. In some embodiments, the frequency with which biological samples may be collected from the subject may be based on the type of disease the subject is being monitored for (e.g., type of cancer), the expected likelihood of recurrence, and the rate of disease progression after recurrence.

Biological samples may be stored using a suitable method. In some embodiments, the biological sample may be stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample may be stored using lyophilization. In some embodiments, a biological sample may be placed into a container that already contains a preservant (e.g., RNALater to preserve RNA) and then frozen (e.g., by snap-freezing), after the collection of the biological sample from the subject. In some embodiments, such storage in frozen state may be done immediately after collection of the biological sample. In some embodiments, a biological sample may be kept at either room temperature or 40C for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acid Citrate Dextrose (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample may be stored at a temperature that preserves stability of the biological sample. In some embodiments, the sample may be stored between 18 and 28° C. (e.g., 25° C.). In some embodiments, the sample may be stored under refrigeration (e.g., 4° C.). In some embodiments, the sample is stored under freezing conditions (e.g., −20° C.). In some embodiments, the sample may be stored under ultralow temperature conditions (e.g., −50° C. to −800° C.). In some embodiments, the sample may be stored under liquid nitrogen (e.g., −1700° C.). In some embodiments, a biological sample may be stored at −60° C. to −80° C. (e.g., −70° C.) for up to 5 years. In some embodiments, a biological sample may be stored at −60° C. to −80° C. (e.g., −70° C.) for up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years. In some embodiments, a biological sample may be stored as described by any of the methods described herein for up to up to 5 years, up to 10 years, up to 15 years, or up to 20 years.

### Sequencing Apparatus

As shown in FIG. 1, the biological sample 102 may be sequenced using a sequencing apparatus 103. In some embodiments, the sequencing apparatus 103 may be any suitable next-generation sequencing apparatus or any high-throughput or massively parallel sequencing apparatus. In some embodiments, the sequencing apparatus 103 may include any suitable sequencing device and/or any sequencing system including one or more devices. In some embodiments, the sequencing apparatus used to sequence the biological sample may be selected from any suitable platform known in the art including, but not limited to, Illumina®, SOLid®, Ion Torrent®, PacBio®, nanopore-based, Sanger sequencing or 454TM. In some embodiments, a sequencing apparatus used to sequence the biological sample is an Illumina sequencing apparatus (e.g., NovaSeq®, NextSeq®, HiSeq®, MiSeq®, or MiniSeq®).

### Sequencing Data

As shown in FIG. 1., the sequence platform 103 processes the sample 102 to generate sequencing data 104.

In some embodiments, sequencing data 104 may comprise sequence reads of polynucleotide sequences from the biological sample of the subject (e.g., the plus strand and the minus strand). In some embodiments, sequence reads may comprise nucleotide representations of the nucleotides of the polynucleotide sequences. In some embodiments, the nucleotide representations may be of any reasonable form, including but not limited to, alphabetic representations, numeric representations, alphanumeric representations, or symbolic representations. In some embodiments, the sequence reads comprise A (representing Adenosine), C (representing Cytosine), G (representing Guanosine), and T (representing Thymidine).

In some embodiments, sequence data comprises sequence reads annotated with additional information (e.g., mapped location, length, sample source, date of acquisition, etc.). In some embodiments sequence reads are mapped to a reference sequence (e.g., a reference genome), thereby providing mapped sequence reads. Accordingly, in some embodiments, sequence data comprises a plurality of annotated sequence reads. Annotated sequence reads and/or sequence data may be provided in a suitable digital format (e.g., a BAM file).

In some embodiments, the sequencing data may comprise sequence reads of any suitable polynucleotide of the biological sample. In some embodiments, the sequencing data comprises sequence reads of RNA of the biological sample. In some embodiments, the sequencing data comprises sequence reads of DNA of the biological sample. In some embodiments, the sequencing data comprises sequence reads of tumor DNA of the biological sample. In some embodiments, the sequencing data comprises sequence reads of cell free DNA (e.g., from healthy cells and cancerous cells (e.g., ctDNA)). In some embodiments, the sequencing data comprises sequence reads of circulating tumor DNA (ctDNA) of the biological sample. In some embodiments, the sequencing data comprises sequence reads of whole exome sequencing of the biological sample. In some embodiments, the sequencing data comprises sequence reads of whole genome sequencing of the biological sample. In some embodiments, the sequencing data comprises sequence reads that cover positions being monitored for mutations (e.g., positions associated with MRD). In some embodiments, the sequencing data comprises sequence reads that were obtained using a targeted gene sequencing panel.

It should be understood that a sequence read is data generated by sequencing polynucleotides (e.g., by using a sequencing apparatus). As such, a sequence read does not include a physical molecule but data representing the same. Thus, a reference to a nucleotide in a sequence read is a reference to information about a nucleotide (e.g., information representing the type of nucleotide—for example “A′, or “G”, or “C” or “T”).

In some embodiments, a sequence read comprises a plus strand primer binding sequence and a minus strand primer binding sequence. In some embodiments, the plus strand primer binding sequence and the minus strand primer binding sequence may be complementary to primers that are used to amplify the polynucleotide. In some embodiments, a plus strand primer binding sequence and a minus strand primer binding sequence are determined (e.g., when generating sequencing data) for use in designing a plus strand sequence primer and a minus strand sequence primer for amplifying a specific polynucleotide (e.g., a polynucleotide comprising a position being monitored for mutations). For example, for each polynucleotide comprising a position being monitored for mutations, a plus strand primer binding sequence and a minus strand primer binding sequence may be determined, where the plus strand primer binding sequence and the minus strand primer binding sequence flank the position being monitored for mutations (e.g., 3′ and 5′ to the position).

In some embodiments, the plus strand primer binding sequence is within 50 nucleotides, within 40 nucleotides, within 30 nucleotides, within 20 nucleotides, within 10 nucleotides, or within 5 nucleotides of 3′ end of the sequence read. In some embodiments, the plus strand primer binding sequence is within 50 nucleotides, within 40 nucleotides, within 30 nucleotides, within 20 nucleotides, within 10 nucleotides, or within 5 nucleotides of 5′ end of the sequence read. In some embodiments, the minus strand primer binding sequence is within 50 nucleotides, within 40 nucleotides, within 30 nucleotides, within 20 nucleotides, within 10 nucleotides, or within 5 nucleotides of 3′ end of the sequence read. In some embodiments, the minus strand primer binding sequence is within 50 nucleotides, within 40 nucleotides, within 30 nucleotides, within 20 nucleotides, within 10 nucleotides, or within 5 nucleotides of 5′ end of the sequence read. In some embodiments, the plus strand primer binding sequence and the minus strand primer binding sequence are 15-30, nucleotides in length.

In some embodiments, a target gene sequencing panel may be used to specifically sequence only certain polynucleotides from the biological sample (e.g., polynucleotides comprising positions being monitored for mutations). In some embodiments, target gene sequencing panels may include representations of polynucleotide sequences that comprise positions being monitored for mutations. In some embodiments, the representations of polynucleotide sequences may be used to determine polynucleotides for amplification (e.g., using the polymerase chain reaction (PCR))). In some embodiments, amplification may be accomplished using PCR primers that are complementary to a polynucleotide comprising positions being monitored for mutations (e.g., a plus strand sequence primer and a minus strand sequence primer). In some embodiments, the plus strand sequence primer is complementary to a plus strand primer binding sequence corresponding to the polynucleotide comprising positions being monitored for mutations. In some embodiments, the minus strand sequence primer is complementary to a minus strand primer binding sequence corresponding to the polynucleotide comprising positions being monitored for mutations. Methods for designing primers for amplification of specific polynucleotides are well known in the art e.g., as described in Untergasser, Andreas et al., Nucleic Acids Research 40.15 (2012): e115-e115. In some embodiments, primers are designed using the ArcherDx panel design algorithm. The primers may be designed using other suitable panel design algorithms and may be used without departing from the scope of the invention.

In some embodiments, the amplification method used with the targeted gene sequencing panel is Anchor-multiplex PCR (AMP). AMP is a multiplex-PCR enrichment chemistry that incorporates strand specific priming and the incorporation of unique molecular identifiers (UMIs) into sequenced reads and is well known in the art, e.g., as described in Zheng Z, et al. Nature medicine 20.12 (2014): 1479-1484.

### Positions Being Monitored for Mutations

As shown in FIG. 1., the indication of positions being monitored for mutations 104 are inputted into the computer 105 and the software 108. In some embodiments, the indication of positions being monitored for mutations may comprise representation of polynucleotides that comprise disease associated mutations (e.g., cancer associated mutations). In some embodiments, the disease associated mutations may be positions being monitored for mutations. In embodiments, positions being monitored for mutations are a predetermined set of positions in a reference genome (e.g., hg19) or portion thereof. In some embodiments, the positions being monitored for mutations are positions in a reference sequence where the reference sequence may be completely arbitrary, it may be a reference genome assembly, or a custom reference. In some embodiments, the positions being monitored for mutations correspond to positions that are mutated in the genome of the tumor of the subject. In some embodiments, positions being monitored for mutations may be positions that are associated with MRD. In some aspects, positions being monitored for mutations may be positions that are correlative with MRD. In some embodiments, the positions being monitored for mutations may be monitored to determine an indication of MRD.

In some embodiments, the positions being monitored for mutations may be determined by methods that are well known in the art. In some embodiments, positions being monitored for mutations may be determined prior to determining an indication of MRD. In some embodiments, determining the positions being monitored for mutations comprises sequencing polynucleotides of a diseased cell from a subject. In some embodiments, determining the positions being monitored for mutations comprises sequencing of polynucleotides of a cancer cell from a subject. In some embodiments, determining the positions being monitored for mutations comprises sequencing a tumor from a subject. In some embodiments, determining the positions being monitored for mutations comprises sequencing a primary tumor from a subject. In some embodiments, determining the positions being monitored for mutations comprises sequencing a secondary tumor from a subject. In some embodiments, determining the positions being monitored for mutations comprises sequencing ctDNA from a subject prior to treatment (e.g., pre-operative). Thus, in some embodiments, the positions being monitored for mutations may be specific to a given subject. In some embodiments, the positions being monitored for mutations may be determined using the ArcherDx panel design algorithm. Other suitable panel design algorithms may be used without departing from the scope of the invention.

In some embodiments, the number of positions being monitored for mutations comprises at least 10 positions, at least 25 positions, at least 50 positions, at least 75 positions, at least 100 positions, at least 125 positions, at least 150 positions, at least 175 positions, at least 200 positions, at least 250 positions, or at least 300 positions. In some embodiments, the number of positions being monitored for mutations may be 10-200 positions. In some embodiments, the number of positions being monitored for mutations may be 25-200 positions. In some embodiments, the number of positions being monitored for mutations may be 50-200 positions. In some embodiments, the number of positions being monitored for mutations may be 75-200 positions. In some embodiments, the number of positions being monitored for mutations may be 100-200 positions. In some embodiments, the number of positions being monitored for mutations may be 10-150. In some embodiments, the number of positions being monitored for mutations may be 25-150 positions. In some embodiments, the number of positions being monitored for mutations may be 50-150 positions. In some embodiments, the number of positions being monitored for mutations may be 75-150 positions. In some embodiments, the number of positions being monitored for mutations may be 100-150 positions.

As shown in FIG. 1, the computing device 105 and software 108 obtain the sequencing data, obtain an indication of the positions being monitored for mutations, and determine an indication of MRD. In some embodiments, computing device 105 may be one or multiple computing devices of any suitable type. In some embodiments, the computing device 105 may be, but is not limited to, a laptop, a desktop, a cloud computer, a server, a phone, or a tablet. In some embodiments, the computing device 105 may be located in a single physical location or located across multiple physical locations. In some embodiments, the computing device 105 may be located in a facility operated by an entity (e.g., a hospital or a research institution). In some embodiments, the software 105 may determine an indication of MRD based on trinucleotide contexts (TNCs) in sequence reads of the sequencing data.

In some embodiments, the computing device 105 may be operated by a user such as a researcher, patient, doctor, clinician, or other individual. For example, the user may provide the sequencing data 104 as input to the computing device 105 (e.g., by uploading a file).

### Trinucleotide Context (TNC)

In some embodiments, a TNC may be a series of three sequential nucleotides in a sequence read. Generally nucleotides in sequence reads may include, but are not limited to, A (representing Adenine), C (representing Cytosine), G (representing Guanine), and T (representing Thymine). In some embodiments, a trinucleotide context may be any permutation of any three of A, C, T, G. For example, a TNC may be, but is not limited to, any one of GAT, TTC, TAT, CAT, TTT and TAA. In some embodiments, sequence reads comprise a plurality of TNCs. In some embodiments, a given nucleotide representation associated with a given position that is included in a first TNC may not be included in a second TNC. In some embodiments, TNCs correspond to amino acid codons and/or anti-codons. In some embodiments, TNCs may be used to determine the first value indicative of the expected number of mutations present in the sequencing data due to sequencing error, which in turn may be used when determining an indication of MRD. One advantage of using TNCs to calculate error rates over using single positions is that error rates can be better estimated when more data is available, e.g., the error of three nucleotide in a TNC compared to the error in a single nucleotide (Deng, Shibing, et al. BMC Bioinformatics 19.1 (2018): 1-7).

### Minimum Residual Disease (MRD)

Minimum residual disease refers to any remaining disease (e.g., diseased cell or ctDNA) that may be present in a subject after the subject has received or completed a treatment for the disease. For example, minimum residual disease associated with cancer may be present when cancer cells, cancer RNA, and/or circulating tumor DNA (e.g., ctDNA) are present in a subject after treatment. In some embodiments, MRD may be detected based on ctDNA detection before cancer relapse is detected using standard surveillance imaging (e.g., computerized tomography (CT), magnetic resonance imaging (MRI), or Positron Emission Tomography (PET)). Some cancer types may shed DNA, which may end up in the bloodstream of the subject (e.g., ctDNA). Thus, in some embodiments, minimum residual disease may be monitored based on sequencing of ctDNA from blood-based biological samples (e.g., plasma). In some embodiments, an indication of minimum residual disease may have increasing likelihood or probability overtime. For example, because cancer cells that survive treatment may continue to replicate and/or metastasize, which may result in additional ctDNA shedding.

### Indication of Minimum Residual Disease (MRD)

Any of the techniques described herein may be used to determine an indication of MRD. In some embodiments, an indication of MRD may include any information that provides an estimate of a likelihood and/or probability that MRD may be present. For example, an indication of MRD may be an alphabetic, numeric, symbolic, or alphanumeric representation of the likelihood and/or probability that MRD is present. In a further example, an indication of MRD may be based on a scale from 0-1, wherein a zero indicates the lowest likelihood or probability that a subject may have MRD and 1 indicates the highest likelihood or probability that a subject may have MRD. In some embodiments, the indication that a subject has MRD may be binary (e.g., “yes” or “no”; “True” or “False”; etc.). In some embodiments, an indication of MRD may be instructions to perform additional tests to confirm the indication of MRD. In some embodiments, an indication of MRD may be based on sequencing data of more than one biological sample from the same subject. For example, an indication of MRD may be determined when analysis of sequencing data from at least two biological samples from the subject both reveal the same result. In another example, an indication of MRD may be determined when analysis of sequencing data from at least one of two or more biological samples from the subject indicate MRD.

In some embodiments, an indication of MRD may be an estimation of the likelihood and/or probability that MRD is present in the biological sample of a subject based on a statistical test that compares the sequencing error (e.g., first value) in the analysis of the biological sample to the actual number of mutations at positions being monitored for mutations (e.g., second value) found in the biological sample. In some embodiments, an indication of minimum residual disease may be based on the number of positions being monitored for mutations.

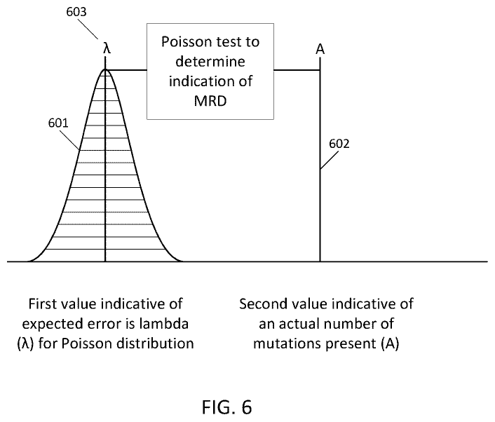

In some embodiments, a statistical test may be used to determine an indication of MRD. In some embodiments, the statistical test may be selected from the group consisting of a Poisson test, a Binomial test, a T-Test, or any other suitable statistical test. In some embodiments, the statistical test may be a non-parametric test (e.g., a Wilcoxon rank-sum test). In some embodiments, the statistical test may be a one-sided test. In some embodiments, the statistical test may be a one-sided Poisson test. In some embodiments, the statistical test may compare a null distribution based on the first value and an alternative hypothesis based on the second value. In some embodiments, an indication of MRD may be based on the p-value of the statistical test being less than a predetermined value alpha. In some embodiments, the alpha may be at most 0.2, at most 0.15, at most 0.1, at most 0.05, at most 0.01, at most 0.005, or at most 0.001. In some embodiments, the alpha is 0.2, 0.15, 0.1, 0.05, 0.01, 0.005 or 0.001. In some embodiments, the alpha may be 0.01. Aspects of determining an indication of MRD are described herein including with reference to FIG. 6.

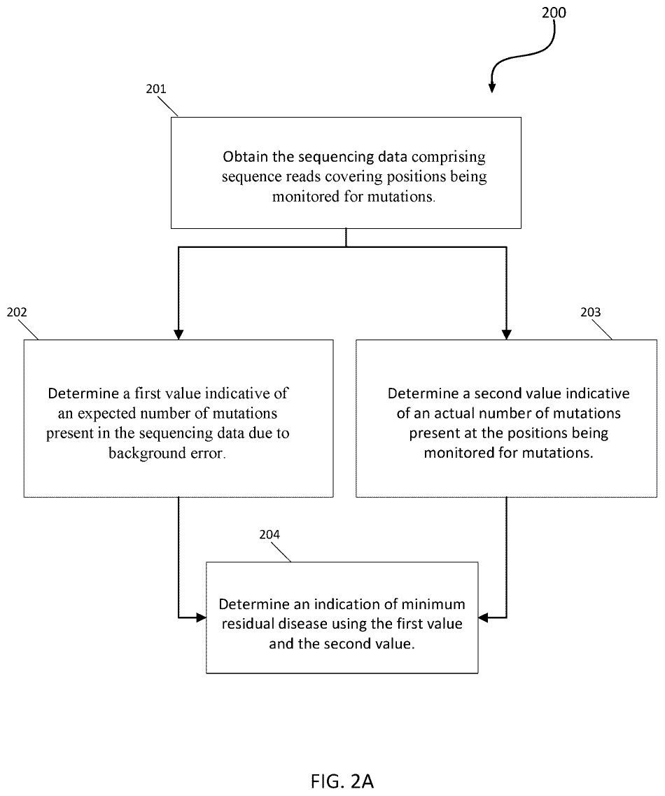

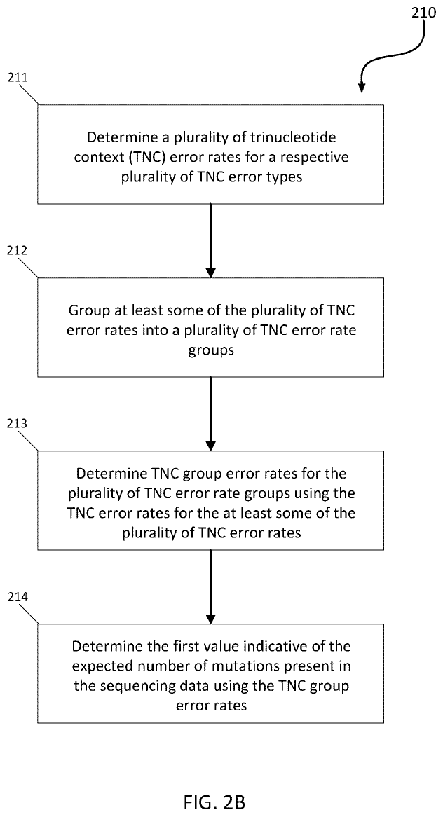

FIG. 2A depicts a flowchart of an illustrative process 200 for determining an indication of minimum residual disease (MRD) based on sequencing data from a biological sample of the subject. Process 200 has the following acts: act 201, obtain the sequencing data comprising sequence reads covering positions being monitored for mutations; act 202, determine a first value indicative of an expected number of mutations present in the sequencing data due to background error; act 203, determine a second value indicative of an actual number of mutations present at the positions being monitored for mutations, and act 204, determine an indication of minimum residual disease using the first value and the second value. In some embodiments, process 200 is embodied in program code (e.g., processor executable instructions) part of software 108 and performed using the computing device 105.

As shown in FIG. 2A, process 200 begins at act 201 where sequencing data is obtained. In some embodiments, the sequencing data may be generated from a biological sample collected from a subject (e.g., a subject having received treatment for cancer), as described above. In some embodiments, the sequencing data comprises sequence reads covering positions being monitored for mutations. In some embodiments, sequencing data may be obtained from a datastore, cloud storage, compact disc, external storage, sequencing instrument, public depository, desktop computer, laptop computer, phone, tablet, flash drive, or any other suitable source. In some embodiments, the sequencing data may be obtained by generating the sequencing data using a sequencing system (e.g., sequencing a biological sample of the subject). Aspects of sequencing data are described herein including with reference to FIG. 1.

In some embodiments, low quality sequence reads may be identified in the sequencing data prior to determining the first value and the second value. In some embodiments, low quality sequence reads may be identified and removed from the sequencing data prior to determining the first value and the second value Methods of removing low quality sequence reads from sequences data are well known in the art, e.g., as described in Chen, Shifu, et al. BMC Bioinformatics 18.3 (2017): 91-100.

In some embodiments, after act 201 and before acts 202 and 203, consensus sequence reads may be generated. In some embodiments, consensus sequence reads may be generated based on unique molecular identifier (UMIs) associated with the consensus sequence reads. UMI's may be a plurality of unique nucleotide sequences that may be ligated to polynucleotide fragments (e.g., DNA fragments from a biological sample) to detect sample preparation bias. Prior to sequencing, polynucleotides extracted from biological samples (e.g., ctDNA) are often amplified using polymerase chain reaction to produce enough material for sequencing. However, the polymerase chain reaction may create amplification bias because some polynucleotides may be amplified with greater efficiency than other polynucleotides. To control for this, UMIs may be ligated to the polynucleotides prior to amplification. During amplification, each polynucleotide-UMI molecule may be copied numerous times, and the UMI may indicate which copies came from the same original polynucleotide regardless of the number of copies that are made. The polynucleotide-UMI may be sequenced producing sequence reads. In some embodiments, consensus sequence reads may be generated by aligning all of the sequence reads having the same UMI and generating a single consensus sequence read. Methods of generating consensus sequence reads are well known in the art, e.g., as described in Chen, Shifu, et al. BMC Bioinformatics 20.23 (2019): 1-8. In some embodiments, each of the consensus sequence reads may be generated from at least a threshold number of sequence reads that are associated with a respective common UMI. In some embodiments, the threshold number of sequence reads that are associated with a respective common UMI may be 2, 3, 4, 5, 6, 7, 8, 9, 10, 11, 12, 13, 14, 15, 16, 17, 18, 19, 20 or any other suitable threshold.

In some embodiments, a subset of the consensus sequence reads may be selected prior to act 202 and act 203. In some embodiments, the subset of consensus sequence reads may be selected to promote use of high quality data when calculating the first value and the second value. In some embodiments, selecting the subset of sequence reads may be based on a measure of similarity (e.g., complementarity) between the plus strand consensus sequence read and the minus strand consensus sequence reads. In some embodiments, selecting the subset may be performed based on relative numbers of plus strand consensus sequence reads and corresponding minus strand consensus sequence reads. For example, a subset of sequence reads may be selected if no more than 95% of the reads associated with a given sequence come from the plus strand consequence sequence read or the minus strand consensus sequence read. In some embodiments, selecting the subset may be performed based on relative numbers of read 1 consensus sequence reads and corresponding read 2 consensus sequence reads. The skilled person will understand that some types of sequencing technologies (e.g., Illumina®) may sequence a given polynucleotide (e.g., a plus strand or a minus strand) from 3′ end and 5′ end of the polynucleotide and thus produce two sequence reads, read 1 and read 2.

Next, process 200 proceeds to acts 202 and 203. As shown in FIG. 2A, these acts may be performed in parallel. However, this need not be the case, as in some embodiments these acts may be performed sequentially (e.g., either 202 is performed prior to 203 or 203 is performed prior to 202), as aspects of the technology described herein are not limited in this respect. Notwithstanding, for clarity, act 202 is described first.

In act 202, the computing device(s) executing process 200 determines a first value indicative of an expected number of mutations present in the sequencing data due to sequencing error. One example of a first value indicative of an expected number of mutations is a first value representing an expected number of mutations. In another example, a first value indicative of an expected number of mutations is a first value corresponding to an expected number of mutations present in the sequencing data. In some embodiments, determining a first value may include, but is not limited to, determining an indication of the error rate of observing mutations at positions being monitored for mutations. In some embodiments, the first value is determined using a subset of sequence reads of the sequencing data.

The first subset of sequence reads, may include one, some, or all of the sequence reads. In some embodiments, the first subset of sequence reads comprises sequence reads that cover positions being monitored for mutations. It is to be understood that sequence reads covering positions being monitored for mutations may also cover background regions. In some embodiments, the first subset of sequence reads consist of reads that have been selected based on the data quality filters described herein. In some embodiments, the first subset of sequence reads comprises reads associated with a position that, when mutated, is indicative of MRD. In some embodiments, the first subset of sequence reads may be the same as the second subset of sequence reads.

Aspects of determining the first value are further described herein including with reference to FIG. 2B. In act 203, the computing device(s) executing process 200 determines a second value indicative of an actual number of mutations present at the positions being monitored for mutations. In some embodiments, the second value may be determined based on the number of times a mutation is observed at a position being monitored for mutations. In some embodiments, the second value may be determined based on the number of times mutations are observed at a plurality of positions being monitored for mutations. For example, the second value may be determined based on the number of times mutations are observed at at least 10 positions being monitored for mutations. In other examples, the second value may be determined based on the number of times mutations are observed at at least 16 positions, at least 25 positions, at least 50 positions, at least 75 positions, at least 100 positions, at least 125 positions, at least 150 positions, at least 175 positions, at least 200 positions, at least 250 positions, or at least 300 positions. In some embodiments, the second value may be determined based on the number of times mutations are observed at 10-200, 16-200, 25-200, 50-200, 75-200, 100-200, 125-200, 150-200, 75-200, 25-150, 50-150, 75-150, 100-150, 25-100, 50-100, 75-100 positions being monitored for mutations. In some embodiments, the second value may be determined based on the number of times mutations are observed at 16-100 positions being monitored for mutations. In some embodiments, the second value may be determined based on the number of times mutations are observed at 50-100 positions being monitored for mutations. In some embodiments, the second value may be determined based on the number of times mutations are observed at 50-200 positions being monitored for mutations. In some embodiments, determining the second value comprises counting the number of mutations present at each position being monitored for mutations. In some embodiments, determining the second value may be based on the number of mutations present at each position being monitored for mutations. In some embodiments, determining the second value may be based on the frequency of mutations present at each position being monitored for mutations. In some embodiments, determining the second value may be performed using consensus sequence reads. Aspects of consensus reads sequences are further described herein.

In some embodiments, the second value may be determined based on a second subset of sequence reads. The second subset of sequence reads, may include one, some, or all of the sequence reads. In some embodiments, the second subset of sequence reads only comprises sequence reads that cover positions being monitored for mutations. In some embodiments, reads covering positions being monitored for mutations may also cover background regions. In some embodiments, the second subset of sequence reads consists of reads that have been selected based on the data quality filters described herein. In some embodiments, the second subset of sequence reads comprising reads associated with position, that when mutated, is indicative of MRD. In some embodiments, the second subset of sequence reads may be the same as the first subset of sequence reads. In some embodiments, the second value is based on the number of times a position being monitored for mutations is mutated. In some embodiments, the second value is the sum of the number of times at least some of the positions being monitored for mutations are mutated. In some embodiments, the second value is the sum of the number of times each position being monitored for mutations is mutated.

After acts 202 and 203 are completed, process 200 proceeds to act 204 where the information determined during those acts (e.g., the first and second values) may be used to determine an indication that the subject has MRD. Examples of determining an indication that the subject has MRD are provided herein including above in the section called Indication of Minimal Residual Disease (MRD) and with reference to FIG. 6.

FIG. 6 illustrates aspects of using a statistical test (e.g., a one-sided Poisson test) to determine a likelihood that MRD may be present using the first value (computed at act 202 and indicative of an expected number of mutations present due to sequencing error) and the second value (computed at act 204 and indicative of the actual number of mutations present). In some embodiments, using the statistical test may comprise determining the likelihood of observing the second value (indicative of the actual number of mutations present) assuming that the distribution of mutations due to sequencing error follows a distribution associated with the null hypothesis of the statistical test. That distribution may be parameterized by one or more parameters. For example, the distribution may be parameterized by one or more parameters indicative of the expected number of mutations present in the sequencing data due to sequencing error. For example, in some embodiments, the distribution may be a Poisson distribution and its mean value (λ) may be set to the first value indicative of an expected number of mutations present in the sequencing data.

For example, as shown in FIG. 6, a one-sided Poisson test may be used to determine the likelihood of an observation 602 (e.g., the actual number of observations present) under the null hypothesis that the expected number of mutations present is distributed according to a Poisson distribution (601) having its mean (2) 603 set to the first value (computed at act 202 of process 200) that is indicative of the expected number of mutations due to sequencing error. In some embodiments, a determination the MRD is present may be made when the p-value for the statistical test is less than a threshold (e.g., 0.1, 0.01, 0.001, 0.0001, etc.).

FIG. 2B describes a flowchart of an illustrative process 210 for determining an expected number of mutations due to sequencing error. Process 201 comprises the following acts: act 211, determine a plurality of trinucleotide context (TNC) error rates for a respective plurality of TNC error types; act 212, group at least some of the plurality of TNC error rates into a plurality of TNC error rate groups; act 213, determine TNC group error rates for the plurality of TNC error rate groups using the TNC error rates for the at least some of the plurality of TNC error rates; and act 214, determine the first value indicative of the expected number of mutations present in the sequencing data using the TNC group error rates.

At act 211 of process 210 the computing device determines a plurality of trinucleotide context (TNC) error rates for a respective plurality of TNC error types. Examples of trinucleotide contexts are provided herein including above in the section called “Trinucleotide Context (TNC)”. In some embodiments, a TNC error rate may be based on the number of times a TNC error type is observed in the sequencing data. In some embodiments, a TNC error rate may be based on the frequency with which a TNC error type is observed in the sequencing data. In some embodiments, determining, using the first subset of sequence reads, a plurality of TNC error rates for a respective plurality of TNC error types comprises determining a frequency of occurrence of each of the TNC error types in the first subset of sequence reads.

In some embodiments, a TNC error type may be a mutated variant of a wildtype TNC. In some embodiments, a wildtype TNC is a TNC that may be naturally occurring at a given position in a genome or a portion thereof (e.g., a reference genome like GRCh38 or hg19). In some embodiments, a mutated variant TNC of a wildtype TNC may be a TNC having a mutation, relative to the wildtype TNC, in at least one position of the TNC. For example, if the reference genome comprised the following sequence 367-ATGGTACTGCGTACG-381, the wildtype TNC at positions 367-369 would be ATG. In this example, the TNC error types of ATG may be, but are not limited to, TTG, AAG, and ATC. In some embodiments, a TNC error type may be dependent on the wildtype TNC. For example, a TNC error type of ATG to AAG may be distinct from a TNC error type of ACG to AAG despite the wildtype TNC having the same sequence for each of mutated variants. In some embodiments, the TNC error types correspond to a mutation in any position of a given TNC. In some embodiments, a TNC error type comprises a mutation in the middle positions of the TNC and does not comprise mutations in the first position or the third position of the TNC (middle position TNC error type). Aspects of middle position TNC error types are described herein including in reference to FIG. 3. In some embodiments, TNC error rates may be determined for a respective plurality of TNC error types.

FIG. 3 illustrates an example of determining TNC error rates. In FIG. 3, consensus sequence reads 304 (described below), all covering the same position being monitored for mutations 313, may be aligned to a reference sequence 303 (e.g., a known sequence comprising the position being monitored for mutations). In some embodiments, the reference sequence is human reference genome assembly hg19. In some embodiments, the reference sequence is human reference genome assembly hg38. In some embodiments, the TNCs of the aligned consensus sequence reads may be identified within predetermined background region(s) 308 and 309. In some embodiments, background region 308 is specific to the minus strand (−) consensus sequences reads and background region 309 is specific to the plus strand (+) consensus sequences reads. Background region 308 is specified by threshold 1 (305) and threshold 3 (307). Background region 309 is specified by threshold 1 (305) and threshold 2 (306). Additional aspects of background regions are discussed below.

In some embodiments, determining TNC error rates comprises counting the number of times the wildtype TNC 300 and the TNC error types 309 occur in the consensus sequence reads. In some embodiments, the counting may be based on the wildtype sequence 301. For each TNC of each consensus sequence read, a determination may be made whether the TNC is a wildtype TNC or a TNC error type by comparing the TNC of the consensus sequence read to the TNC at the same position in the wildtype sequence 301. In some embodiments, counts for each Occurrence 310 of the wildtype TNC and each Occurrence 310 of each TNC error type may be determined 314. In some embodiments, a Total 311 of wild type TNC occurrence and TNC error type occurrences is determined.

In some embodiments, the TNC error types counted are TNC error types 309 having a mutation in the middle position of the TNC (relative to the wildtype TNC), but not having a mutations in the first (5′) or last (3′) positions of the TNC (e.g., middle position TNC error type). For example, middle position TNC error types of ATG may include, but are not limited to, AAG, ACG, and AGG. In some embodiments, there are 192 different middle position TNC error types. The value of 192 different middle TNC error types may be determined as follows: there are 64 possible TNCs based on the four common nucleotides A, T, G, and C because each position of the TNC (first, middle, and last) can have any one of A, T, G, or C in that position (e.g., 4 common nucleotides\hat{ }3 positions). Each TNC's middle position may be mutated in three different ways (e.g., ATG to AAG, ACG or AGG). Thus, in some embodiments, there are 64 TNCs times 3 possible middle positions mutations (e.g., 192 different possible middle position TNC error types).

In some embodiments, a plurality of TNC error types comprises any combination of any number of middle position TNC error types 309. In some embodiments, a plurality of TNC error types comprises 10, 25, 50, 75, 96, 100, 125, 150, 176, 192, or any other reasonable number of TNC error types. In some embodiments, a plurality of TNC error types comprises at least 10, at least 25 TNC error types, at least 50 TNC error types, at least 75 TNC error types, at least 96 TNC error types, at least 100 TNC error types, at least 125 TNC error types, at least 150 TNC error types, at least 176 TNC error types, or at least 192 TNC error types. In some embodiments, a plurality of TNC error types comprises 192 middle position TNC error types. In some embodiments, a plurality of TNC error types refers to 96 middle position TNC error types. In some embodiments, a plurality of TNC error types refers to 10-25, 10-75, 10-125, 10-176, 10-192, 25-50, 25-75, 25-125, 25-176, 25-192, 50-75, 50-125, 50-176, 50-192 TNC error types. In some embodiments, a plurality of TNC error types may be based on the TNC error types present in the sequence reads. In some embodiments, a TNC error rate is determined for each TNC error type of the plurality of TNC error types.

A TNC error rate 312, in some embodiments, may be a function of the Occurrence 310 and the Total 311. In some embodiments, the TNC error rate 312 for a given TNC error type of 309 may be the Occurrence 310 of that TNC error type divided by the Total 311. In some embodiments, the TNC error rate 312 for a given TNC error type of 309 may be determined based on the Occurrence 310 of the TNC error type over all of the same wildtype TNCs and the Total 311 over all of the same wildtype TNCs. For example, a wildtype TNC, “ATG” may occur in two different positions in a wildtype sequence 300, position one and position two. The Occurrence of the wildtype TNC in the consensus sequence reads at position one may be 15 and the Occurrence of the wildtype TNC at position two may be 19. Additionally, the Occurrence of TNC error type “AGG” in the consensus sequence reads may be 2 at position one and 3 at position two. In this example, the TNC error rate may be calculated as (2+3)/(15+19). In some aspects, the TNC error rate 312 for a given TNC error type of 309 may be determined using the following formula:

$$TNC error {rate}^{type} = \frac{{\sum }_{i = 1}^{n} {Occurence}_{i}^{type}}{{\sum }_{i = 1}^{n} {Total}_{i}^{type}}$$

where n is the number of TNCs that correspond to the TNC error type (type) having the same sequence but are in different locations in the background region (e.g., 3-ATGTTTCATTTGATG-17).

In some embodiments, the TNC error rates comprise an error rate for each TNC error type. In some embodiments, the TNC error rates comprise an error rate for each middle position TNC error type. In some embodiments, the TNC error rates comprises 10, 25, 50, 75, 100, 125, 150, 176, 192, or any other reasonable number of TNC error rates. In some embodiments, the TNC error rates comprises 10-25, 10-75, 10-125, 10-176, 10-192, 25-50, 25-75, 25-125, 25-176, 25-192, 50-75, 50-125, 50-176, 50-192, or any other reasonable number of TNC error rates. In some embodiments, a plurality of trinucleotide context (TNC) error rates comprises 192 TNC error rates. In some embodiments, a plurality of trinucleotide context (TNC) error rates comprises 96 TNC error rates. In some embodiments, determining, using the first subset of sequence reads, a plurality of TNC error rates for a respective plurality of TNC error types comprises determining a frequency of occurrence of each of the TNC error types in the background regions of the first subset of sequence reads.

In some embodiments, the sequence reads described herein comprise background regions. Background regions, in some aspects, may identify a region of the sequence that may be used for determining a first value indicative of an expected number of mutations present in the sequencing data due to background error. In some embodiments, the background regions do not comprise any positions being monitored for mutations.

In some embodiments, a background region of the plurality of background regions comprises at least 25, nucleotides at least 50 nucleotides, at least 100 nucleotides, at least 125 nucleotides, at least 150 nucleotides, at least 175 nucleotides, at least 200 nucleotides, at least 250 nucleotides, at least 300 nucleotides, at least 400 nucleotides, at least 500 nucleotides, at least 750 nucleotides, at least 1000 nucleotides, at least 2000 nucleotides, at least 5000 nucleotides, or at least 10000 nucleotides. In some embodiments, the background region comprises at least 400 nucleotides. In some embodiments, the background region comprises at least 10 trinucleotides at least 25 trinucleotides, at least 50 trinucleotides, at least 100 trinucleotides, at least 125 trinucleotides, at least 150 trinucleotides, at least 175 trinucleotides, at least 200 trinucleotides, at least 250 trinucleotides, at least 300 trinucleotides, at least 400 trinucleotides, at least 500 trinucleotides, at least 750 trinucleotides, at least 1000 trinucleotides, at least 2000 trinucleotides, at least 3000 trinucleotides, at least 5000 trinucleotides, or at least 10,000 trinucleotides. In some embodiments, the background region comprises at least 130 trinucleotides.

In some embodiments, the background region may be identified based on a first threshold, a second threshold and/or a third threshold.

In some embodiments, the background region may be identified based on a first threshold. In some embodiments, the first threshold sets a nucleotide distance between a position being monitored for a mutation and the beginning of the background region. In some embodiments, the first threshold is 0 nucleotides, which indicates that the position being monitored for mutations is included in the background region. In some embodiments, the first threshold is 1 nucleotide, which indicates that the position being monitored for mutations is not included in the background region. In some embodiments, the first threshold is 2 nucleotides, which indicates that the position being monitored for mutations and the nucleotides on either side of that position are not included in the background region. In some embodiments, the first threshold is 2, 3, 4, 5, 6, 7, 8, 9, 10, 15, 20, 25, 30, 50, 100, 150, 200 or more nucleotides. In some embodiments, the first threshold may be 2-5, 2-10, 2-20, 2-50, 2-100 or 2-200 nucleotides. In some embodiments, the first threshold may be 5-10, 5-20, 5-50, 5-100 or 5-200 nucleotides. In some embodiments, the first threshold may be 20-50, 20-100 or 20-200 nucleotides.

In some embodiments, the background region may be identified based on a second threshold. In some embodiments, the second threshold may be specific to the plus strand sequence reads. In some embodiments, the second threshold may be a distance from the beginning of the plus strand sequence read (e.g., the plus strand primer binding sequence). For example, if the second threshold is 100 nucleotides, then any nucleotides falling within the first 100 nucleotides of the plus strand sequence reads may be included in the background region. In some embodiments, the second threshold may be 50, 100, 150, 200, 250, 300, or 400 nucleotides. In some embodiments, the second threshold may be at least 50 nucleotides, at least 100 nucleotides, at least 150 nucleotides, at least 200 nucleotides, at least 250 nucleotides, at least 300 nucleotides, or at least 400 nucleotides. In some embodiments, the second threshold may be between 50-100 nucleotides, 100-150 nucleotides, 150-200 nucleotides, 200-250 nucleotides, 250-300 nucleotides, 300-400 nucleotides, or 100-400 nucleotides. In some embodiments, the second threshold may be based on the sequencing quality associated with the nucleotides of the sequence reads. It is known that sequencing near the end of a sequence read can produce lower quality data than sequencing at the beginning of a sequence read. Thus, the skilled person, based on the data quality of the sequence reads, may set a second threshold to remove low quality nucleotide identifications near the end of the sequence read.

In some embodiments, the background region may be identified based on a third threshold. In some embodiments, the third threshold may be specific to the minus strand sequence reads. In some embodiments, the third threshold may be a distance from the beginning of the minus strand sequence read (e.g., at the minus strand primer binding sequence). For example, if the third threshold is 100 nucleotides, then any nucleotides falling within the first 100 nucleotides of the third sequence reads may be included in the background region. In some embodiments, the third threshold may be 50, 100, 150, 200, 250, 300, or 400 nucleotides. In some embodiments, the third threshold may be at least 50 nucleotides, at least 100 nucleotides, at least 150 nucleotides, at least 200 nucleotides, at least 250 nucleotides, at least 300 nucleotides, or at least 400 nucleotides. In some embodiments, the third threshold may be between 50-100 nucleotides, 100-150 nucleotides, 150-200 nucleotides, 200-250 nucleotides, 250-300 nucleotides, 300-400 nucleotides, or 100-400 nucleotides. In some embodiments, the third threshold may be based on the sequencing quality associated with the nucleotides of the sequence reads. It is known that sequencing near the end of a sequence read can produce lower quality data than sequencing at the beginning of a sequence read. Thus, the skilled person, based on the data quality of the sequence reads, may set a third threshold to remove low quality nucleotide identifications near the end of the sequence read. In some embodiments, the first threshold and the second threshold are the same number of nucleotides from their corresponding primer binding sequences.

In some embodiments, after act 211 and prior to act 212, TNC error rates may be selected for grouping. In some embodiments, selecting TNC error rates for grouping may be based on an error rate threshold (e.g., 401 of FIG. 4). For example, in some embodiments, if a TNC error rate does not exceed the error rate threshold then the TNC error rate may be selected for grouping. In some embodiments, the error rate threshold is 0.0001%-1%, 0.001%-0.1%, or 0.005%-0.05%. In some embodiments, the error rate threshold is 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, or 0.09%, 0.1%. In some embodiments, the error rate threshold is 0.01%. In some embodiments, a TNC error rate may be selected if the upper bound of a confidence interval of the TNC error rate is less than the error rate threshold.

In some aspects, FIG. 4 illustrates selecting TNC error rates (e.g., 403 and 405) for grouping based on their respective 99% binomial confidence interval (CI) upper bound (e.g., 402 & 404). In some embodiments, the error rate threshold 401 is a static threshold (e.g., 0.001%-0.10%,). In some embodiments, for each individual TNC error rate (e.g., 402 and 404), a binomial confidence interval 406 (e.g., a 99% confidence interval) is also calculated. In some embodiments, for each individual TNC error rate binomial CI, if the upper bound of the TNC error rate binomial CI (e.g., 404) is less than the threshold 401, then the TNC error rate is selected for grouping. Thus, selecting TNC error rates may allow removal of error rates that are anomalously high (e.g., exceeding the confidence interval 406) and could decrease the accuracy error estimation (e.g., determining the first value).

Next, process 210 proceeds to act 212, “group at least some of the plurality of TNC error rates into a plurality of TNC error rate groups.” In some embodiments, grouping may be performed using any known method of grouping. In some embodiments, grouping may be performed using a clustering algorithm. In some embodiments, the clustering algorithm may be selected from the group consisting of Affinity Propagation, Agglomerative Clustering, Balanced Iterative Reducing and Clustering using Hierarchies (BIRCH), Density-Based Spatial Clustering of Applications with Noise (DBSCAN), K-Means, Mini-Batch K-Means, Mean Shift, Ordering Points To Identify the Clustering Structure (OPTICS), Spectral Clustering, Mixture of Gaussians, and partition around medoids (PAM) clustering. In some embodiments, grouping is performed by a process comprising nearest neighbors clustering or hierarchical agglomerative clustering.

In some embodiments, grouping may be performed using partition around medoids (PAM) clustering.

In some embodiments, the TNC error rates may be grouped into 2, 3, 4, 5, 6, 7, 8, 9, 10, or any other suitable number TNC error rate groups. In some embodiments, a suitable number of TNC error rates groups may be determined based on obtaining a sufficient read depth in each group for the purpose of increasing statistical power. In some embodiments, different TNC error rate groups are associated with different numbers of TNC error rates. For example, group 1 may be associated with 40 TNC error rates and group 2 may be associated with 25 TNC error rates.

Next, process 210 proceeds to act 213, “determine TNC group error rates for the plurality of TNC error rate groups using the TNC error rates for at least some of the plurality of TNC error rates.” In some embodiments, the TNC error rates comprises TNC error rates that were selected, as described above. In some embodiments, the TNC error rates comprise a TNC error rate associated with 10, 25, 50, 75, 96, 100, 125, 150, 176, 192, or any other possible number of TNC error types. In some embodiments, the TNC error rates comprises TNC error rate associated with at least 10 TNC error types, at least 25 TNC error types, at least 50 TNC error types, at least 75 TNC error types, at least 96 TNC error types, at least 100 TNC error types, at least 125 TNC error types, at least 150 TNC error types, at least 176 TNC error types, or at least 192 TNC error types). In some embodiments, the TNC error rates comprise a TNC error rate associated with 192 TNC error types. In some embodiments, the TNC error rates comprises TNC error rate associated with 96 TNC error types. In some embodiments, the TNC error rates comprises TNC error rates associated with 10-25, 10-75, 10-125, 10-176, 10-192, 25-50, 25-75, 25-125, 25-176, 25-192, 50-75, 50-125, 50-176, 50-192 TNC error types. In some embodiments, the TNC error rates comprises TNC error rates based on the TNC error types present in the sequence reads.

In some embodiments, determining a TNC group error rate is based on the TNC error rates associated with the group. In some embodiments, a TNC group error rate may be determined as an average of the TNC error rates associated with the TNC error rate group. In some embodiments, a TNC group error rate may be determined using a weighted average of the TNC error rates associated with the TNC error rate group. In some embodiments, a TNC group error rate may be determined using a population weighted average of the TNC error rates associated with the TNC error rate group. In some embodiments, a TNC group error rate may be determined using a mean or median of the TNC error rates associated with the TNC error rate group.

In some aspects, FIG. 5 illustrates TNC group error rates 501-504 for a respective plurality of TNC error rate groups 509. FIG. 5 shows TNC error rate confidence intervals e.g., 505 and 506 (e.g., 99% binomial confidence intervals) grouped into any one of Groups 1-4. The TNC group error rates 501-504, in some embodiments, may be determined by calculating the population weighted average of each group (501 (μ1), 502 (μ2), 503 (μ3), 504 (μ4)). In some embodiments, the TNC group error rates 501-504 may be determined by calculating the median of each group. In some embodiments, the TNC group error rate may be determined by calculating the mean of each group 501-504.

Next, process 210 proceeds to act 214, “determine the first value indicative of the expected number of mutations present in the sequencing data using the TNC group error rates.” In some embodiments, the first value may be based on at least one of the TNC group error rates. In some embodiments, the first value may be based on each of the TNC group error rates. In some embodiments, the first value may be based on the number of times each position being monitored for mutations is covered by a sequence read. In some embodiments, the first value may be based on the number of times each position being monitored for mutations is covered by a sequence read in each TNC error group. In some embodiments, the first value may be based on a function of the TNC group error rates and the number of times a position being monitored for mutations is covered by a sequence read in each TNC error rate group. In some embodiments, the first value may be based on a linear combination of the TNC group error rates and the number of times each position being monitored for mutations is covered by a sequence read in each TNC error rate group. In some embodiments, the first value may be determined as follows:

$$First value = ( μ 1 \times r 1 ) + ( μ 2 \times r 2 ) … + … ( μ n \times rn )$$

where n may be the total number of TNC error rate groups, μ1 to un may be the TNC group error rates (e.g., population weighted average of the TNC error rates associated with the TNC error rate group) associated with TNC error rate groups 1 to n, and r1 to rn may be the sum of number of times each position being monitored for mutations is covered by a sequence read (both mutated and not mutated positions) in a given TNC error rate group 1 to n. Or in other words, r1 to rn may be the sum of a number of sequences (e.g., consensus sequences) that cover positions being monitored for mutations in a given TNC error rate group 1 to n.

### Computer Implementation

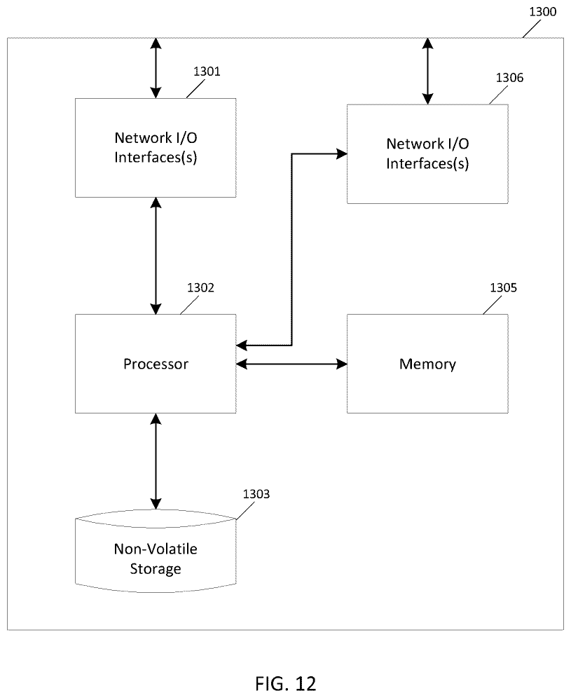

An illustrative implementation of a computer system 1300 that may be used in connection with any of the embodiments of the technology described herein (e.g., such as the methods of FIGS. 2A-2B) is shown in FIG. 12. The computer system 1300 includes one or more processors 1302 and one or more articles of manufacture that comprise non-transitory computer-readable storage media (e.g., memory 1305 and one or more non-volatile storage media 1303). The processor 1302 may control writing data to and reading data from the memory 1305 and the non-volatile storage device 1303 in any suitable manner, as the aspects of the technology described herein are not limited to any particular techniques for writing or reading data. To perform any of the functionality described herein, the processor 1302 may execute one or more processor-executable instructions stored in one or more non-transitory computer-readable storage media (e.g., the memory 1305), which may serve as non-transitory computer-readable storage media storing processor-executable instructions for execution by the processor 1302.

Computing device 1300 may also include a network input/output (I/O) interface 1301 via which the computing device may communicate with other computing devices (e.g., over a network), and may also include one or more user I/O interfaces 1306, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationships between the fields. However, any suitable mechanism may be used to establish a relationship between information in fields of a data structure, including through the use of pointers, tags or other mechanisms that establish relationships between data elements.

When implemented in software, the software code can be executed on any suitable processor or collection of processors, whether provided in a single computer or distributed among multiple computers. Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The methods illustratively disclosed herein suitably may be practiced in the absence of any element which is not specifically disclosed herein. Thus, for example, in each instance herein the term “comprising” can be replaced with “consisting essentially of” or “consisting of”.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

### EXAMPLES

Example A1. A method for determining whether sequencing data of a biological sample of a subject provides an indication that the subject has minimum residual disease, the method comprising:
using at least one computer hardware processor to perform:
   (A) obtaining the sequencing data, the sequencing data being previously generated by sequencing the biological sample of the subject, the sequencing data comprising sequence reads covering positions being monitored for mutations;
   (B) determining, using at least a first subset of the sequence reads, a first value indicative of an expected number of mutations present in the sequencing data due to sequencing error, the determining comprising:
      determining, using the first subset of sequence reads, a plurality of nucleotide context (NC) error rates for a respective plurality of NC error types;
      grouping at least some of the plurality of NC error rates into a plurality of NC error rate groups;
      determining NC group error rates for the plurality of NC error rate groups using the NC error rates for the at least some of the plurality of NC error rates; and
      determining the first value indicative of the expected number of mutations present in the sequencing data using the NC group error rates;
   (C) determining, using at least a second subset of the sequence reads, a second value indicative of an actual number of mutations present at the positions being monitored for mutations; and
   (D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

Example A2. The method of Example A1, wherein the NC is selected from a single nucleotide context (SNC), dinucleotide context (DNC), trinucleotide context (TNC), four nucleotide context (4NC), five nucleotide context (5NC), six nucleotide context (6NC), seven nucleotide context (7NC) and eight nucleotide context (8NC).

Example A3. The method of example A1 or A2, wherein the sequence reads cover at least 10 positions being monitored for mutations.

Example A4. The method of any one of examples A1 to A3, wherein the sequence reads cover 10-200 positions being monitored for mutations.

Example A5. The method of any one of examples A1-A4, wherein the sequence reads cover 50-200 positions being monitored for mutations.

Example A6. The method of any one of examples A1-A5, further comprising: obtaining the sequencing data by sequencing the biological sample.

Example A7. The method of any one of examples A1-A6, wherein the biological sample is a bodily fluid or a sample obtained from a bodily fluid.

Example A8. The method of any one of examples A1-A7, wherein the sequencing data comprises sequence reads from circulating tumor DNA (ctDNA).

Example A9. The method of any one of examples A1-A8, wherein each of the sequence reads covers at least one of the positions being monitored for mutations.

Example A10. The method of any one of examples A1-A9, wherein the sequence reads were obtained using whole exome sequencing.

Example A11. The method of any one of examples A1-A11, wherein the sequence reads were obtained using a targeted gene sequencing panel.

Example A12. The method of example A11, wherein the targeted gene sequencing panel targets sequences covering positions being monitored for mutations.

Example A13. The method of example A11, wherein primers are used to amplify the sequences comprising positions being monitored for mutations.

Example A14. The method of example A11, wherein the sequences targeted by the targeted gene sequencing panel were determined using sequence data from a primary tumor of the subject.

Example A15. The method of any one of examples A1-14, wherein the first subset of the sequence reads and the second subset of the sequence reads are the same.

Example A16. The method of any one of examples A1-A15, wherein (B) is performed using at least the first subset of the sequence reads and one or more sequence reads in the sequencing data that do not cover the positions being monitored for mutations.

Example A17. The method of any one of examples A1-A16, wherein performing (B) further comprises:
generating consensus sequence reads using at least the first subset of the sequence reads,
wherein each of the consensus sequence reads is generated from those sequence reads, in at least the first subset of the sequence reads, that are associated with a respective common unique molecular identifier (UMI),
wherein determining the plurality of NC error rates for the respective plurality of NC error types is performed using the generated consensus sequence reads.

Example A18. The method of example A17, wherein each of the consensus sequence reads is generated from at least a threshold number of sequence reads that are associated with a respective common UMI.

Example A19. The method of example A18, wherein the threshold number of sequence reads is between 2 and 20.

Example A20. The method of any one of examples A17 to A19, further comprising:
selecting a subset of the consensus sequence reads,
wherein determining the plurality NC error rates for the respective plurality of NC error types is performed using only the selected subset of consensus sequence reads.

Example A21. The method of example A20, wherein the consensus sequence reads comprise plus strand consensus sequence reads and minus strand consensus sequence reads, and wherein selecting the subset is performed using a criterion that applies a measure of similarity between corresponding plus strand consensus sequence reads and minus strand consensus sequence reads.

Example A22. The method of example A20, wherein the consensus sequence reads comprise plus strand consensus sequence reads and minus strand consensus sequence reads, and selecting a subset of the consensus reads using one or more criteria that apply to the plus strand consensus sequence reads and minus strand consensus sequence reads.

Example A23. The method of any one of examples A20-A22, wherein the consensus sequence reads comprise plus strand consensus sequence reads and minus strand consensus sequence reads, and wherein selecting the subset is performed using a criterion that applies to the relative numbers of plus strand consensus sequence reads and corresponding minus strand consensus sequence reads.

Example A24. The method of any one of examples A17-A23, wherein determining the plurality of NC error rates for the respective plurality of NC error types using the consensus sequence reads comprises:
determining the plurality of NC error rates using background regions of the consensus sequence reads,
wherein the positions being monitored for mutations include a first position,
wherein the consensus sequence reads include a first consensus sequence read that covers the first position and the background regions include a first background region for the first consensus sequence read,
wherein the first background region comprises nucleotides in the first consensus sequence read that are at least a first threshold distance away from the first position.

Example A25. The method of example A24, wherein background regions do not include the positions being monitored for mutations.

Example A26. The method of any one of examples A17-A23,
wherein the consensus sequence reads comprise, for a first position of the positions being monitored for mutations, a first group of plus strand consensus sequence reads associated with a plus strand primer binding sequence at 3′ terminal of each of the plus strand consensus sequence reads in the first group and a second group of minus strand consensus sequence reads associated with a minus strand primer binding sequence at 3′ terminal of the minus strand consensus sequence reads in the second group,
wherein determining the plurality of NC error rates for the respective plurality of NC error types using the consensus sequence reads comprises:
   determining the plurality of NC error rates using:
      nucleotides, in any sequence read in the first group of plus strand consensus sequence reads, which are located within a second threshold distance of the plus strand primer binding sequence, and
      nucleotides, in any sequence read in the second group of minus strand consensus sequence reads, which are located within a third threshold distance of the minus strand primer binding sequence.

Example A27. The method of any one of examples A17-A26, wherein determining the plurality of NC error rates using the consensus sequence reads comprises determining a frequency of occurrence of each of the TNC error types in the consensus sequence reads.

Example A28. The method of example A21, wherein determining the plurality of NC error rates for the respective plurality of NC error types using the consensus sequence reads comprises:
determining the plurality of NC error rates from background regions of the consensus sequence reads,
wherein the consensus sequence reads include a first consensus sequence read and the background regions include a first background region for the first consensus sequence read,
wherein the NC error rates are determined based on how often each of the NC error types occurs in the first background region for the first consensus sequence read.

Example A29. The method of any one of examples A1-A28, wherein NC error types correspond to a mutation in any position of a given NC.

Example A30. The method of any one of examples A1-A29, wherein each of the NC error types corresponds to a specific mutation of a middle nucleotide in a given NC.

Example A31. The method of any one of examples A1-A30, further comprising:
after determining the plurality of NC error rates and before
   grouping at least some of the plurality of NC error rates into a plurality of NC error rate groups, determining confidence intervals for the NC error rates; and selecting the at least some of the plurality of NC error rate for grouping using a criterion that applies to the confidence intervals for the NC error rates.

Example A32. The method of any one of examples A1-A31, wherein grouping at least some of the plurality of NC error rates into a plurality of NC error rate groups comprises clustering the plurality of NC error rates.

Example A33. The method of any one of examples A1-A32, wherein grouping at least some of the plurality of NC error rates into a plurality of NC error rate groups comprises grouping using partition around medoids (PAM) clustering.

Example A34. The method of any one of examples A1-A33, wherein grouping at least some of the plurality of NC error rates comprises grouping into 4 NC error rate groups.

Example A35. The method of any one of examples A1-A34, wherein determining the first value indicative of the expected number of mutations present in the sequencing data is performed using at least some of the NC group error rates and the number of times at least some of the positions being monitored for mutations are covered by a sequence read in the first subset of sequence reads.

Example A36. The method of any one of examples A1-A35, wherein determining the first value indicative of the expected number of mutations present in the sequencing data comprises:
determining the first value as a weighted linear combination of the NC error group rates with each particular one of the NC error group rates being weighted by a number of times a position being monitored is covered by a sequence read, in the first subset of sequence reads, corresponding to a NC error type that belongs to that particular NC error group.

Example A37. The method of any one of example A1-A36, wherein performing (C) further comprises:
generating second consensus sequence reads using at least the second subset of the sequence reads, wherein each of the second consensus sequence reads is generated from those sequence reads, in at least the second subset of the sequence reads, which are associated with a respective common unique molecular identifier (UMI),
wherein determining the second value indicative of an actual number of mutations present at the positions being monitored for mutations is performed using the second consensus sequence reads.

Example A38. The method of any one of examples A1-A37, wherein (D) is performed using a statistical hypothesis test having a null hypothesis, by comparing the second value to a distribution associated with the null hypothesis, wherein the distribution has one or more parameters that depend on the first value.

Example A39. The method of example A38, wherein the distribution is a Poisson distribution having a mean value (2) that is set to the first value.

Example A40. The method of example A38 or example A39, wherein using the statistical hypothesis test comprises determining a measure of likelihood, under the null hypothesis, of observing the actual number of mutations indicated by the second value.

Example A41. The method of any one of examples A1-A40, wherein (D) is performed using a one-sided Poisson hypothesis test.

Example A42. The method of example A41, wherein using the one-sided Poisson hypothesis test comprises: setting a mean value (2) of a Poisson distribution to the first value and determining a measure of likelihood, under the Poisson distribution, of observing the actual number of mutations indicated by the second value.

Example A43. The method of example A42, determining whether the sequencing data provides the indication that the subject has minimum residual disease using the measure of likelihood.

Example A44. The method of any of examples A39-A43, wherein the subject is likely to have minimum residual disease if the second value indicates that the null hypothesis can be rejected.

Example A45. The method of any of examples A1-A44, wherein (D) further comprises: providing the indication that the subject has minimum residual disease.

Example A46. The method of any one of examples A1-A45, further comprising using the at least one computer hardware processor to perform:
obtaining one or more of further sequencing data previously generated by sequencing one or more further biological sample(s) of the subject, each of the one or more of further sequencing data comprising further sequence reads covering the positions being monitored for mutations, and for each of the further sequence reads of the one or more of further sequencing data:
determining, using at least a first subset of the further sequence reads, a further first value indicative of an expected number of mutations present in a respective sequencing data due to sequencing error, the determining comprising:
   determining a further plurality of NC error rates for a respective plurality of NC error types;
   grouping at least some of the further plurality of NC error rates into a further plurality of NC error rate groups;
   determining further NC group error rates for the further plurality of NC error rate groups using the further NC error rates for the at least some of the further plurality of NC error rates; and
   determining the further first value indicative of the expected number of mutations present in the respective sequencing data using the further NC group error rates; determining, using at least a second subset of the further sequence reads, a further second value indicative of an actual number of mutations present at the positions; and determining whether the respective sequencing data provides the indication that the subject has minimum residual disease using the further first value indicative of the expected number of mutations present in the respective sequencing data due to sequencing error and the further second value indicative of the actual number of mutations present in the respective sequencing data at the positions being monitored for mutations.

Example 1. A method for determining whether sequencing data of a biological sample of a subject provides an indication that the subject has minimum residual disease, the method comprising:
using at least one computer hardware processor to perform:
   (A) obtaining the sequencing data, the sequencing data being previously generated by sequencing the biological sample of the subject, the sequencing data comprising sequence reads covering positions being monitored for mutations;
   (B) determining, using at least a first subset of the sequence reads, a first value indicative of an expected number of mutations present in the sequencing data due to sequencing error, the determining comprising:
      determining, using the first subset of sequence reads, a plurality of trinucleotide context (TNC) error rates for a respective plurality of TNC error types;
      grouping at least some of the plurality of TNC error rates into a plurality of TNC error rate groups;
      determining TNC group error rates for the plurality of TNC error rate groups using the TNC error rates for the at least some of the plurality of TNC error rates; and
      determining the first value indicative of the expected number of mutations present in the sequencing data using the TNC group error rates;
   (C) determining, using at least a second subset of the sequence reads, a second value indicative of an actual number of mutations present at the positions being monitored for mutations; and
   (D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

Example 2. The method of example 1, wherein the sequence reads cover at least 10 positions being monitored for mutations.

Example 3. The method of any one of examples 1 to example 2, wherein the sequence reads cover 10-200 positions being monitored for mutations.

Example 4. The method of any one of examples 1-3, wherein the sequence reads cover 50-200 positions being monitored for mutations.

Example 5. The method of any one of examples 1-4, further comprising: obtaining the sequencing data by sequencing the biological sample.

Example 6. The method of any one of examples 1-5, wherein the biological sample is a bodily fluid or a sample obtained from a bodily fluid.

Example 7. The method of any one of examples 1-6, wherein the sequencing data comprises sequence reads from circulating tumor DNA (ctDNA).

Example 8. The method of any one of examples 1-7, wherein each of the sequence reads covers at least one of the positions being monitored for mutations.

Example 9. The method of any one of examples 1-8, wherein the sequence reads were obtained using whole exome sequencing.

Example 10. The method of any one of examples 1-9, wherein the sequence reads were obtained using a targeted gene sequencing panel.

Example 11. The method of example 10, wherein the targeted gene sequencing panel targets sequences covering positions being monitored for mutations.

Example 12. The method of example 10, wherein primers are used to amplify the sequences comprising positions being monitored for mutations.

Example 13. The method of example 10, wherein the sequences targeted by the targeted gene sequencing panel were determined using sequence data from a primary tumor of the subject.

Claims 10-27 relate to (B): determining expected # of mutations due to sequencing error.

Example 14. The method of any one of examples 1-13, wherein the first subset of the sequence reads and the second subset of the sequence reads are the same.

Example 15. The method of any one of examples 1-14, wherein (B) is performed using at least the first subset of the sequence reads and one or more sequence reads in the sequencing data that do not cover the positions being monitored for mutations.

Example 16. The method of any one of examples 1-15, wherein performing (B) further comprises:
generating consensus sequence reads using at least the first subset of the sequence reads,
wherein each of the consensus sequence reads is generated from those sequence reads, in at least the first subset of the sequence reads, that are associated with a respective common unique molecular identifier (UMI),
wherein determining the plurality of trinucleotide context (TNC) error rates for the respective plurality of TNC error types is performed using the generated consensus sequence reads.

Example 17. The method of example 16, wherein each of the consensus sequence reads is generated from at least a threshold number of sequence reads that are associated with a respective common UMI.

Example 18. The method of example 17, wherein the threshold number of sequence reads is between 2 and 20.

Example 19. The method of example 16, further comprising:
selecting a subset of the consensus sequence reads,
wherein determining the plurality of trinucleotide context (TNC) error rates for the respective plurality of TNC error types is performed using only the selected subset of consensus sequence reads.

Example 20. The method of example 19, wherein the consensus sequence reads comprise plus strand consensus sequence reads and minus strand consensus sequence reads, and wherein selecting the subset is performed using a criterion that applies a measure of similarity between corresponding plus strand consensus sequence reads and minus strand consensus sequence reads.

Example 21. The method of example 19, wherein the consensus sequence reads comprise plus strand consensus sequence reads and minus strand consensus sequence reads, and selecting a subset of the consensus reads using one or more criteria that apply to the plus strand consensus sequence reads and minus strand consensus sequence reads.

Example 22. The method of any one of examples 19-21, wherein the consensus sequence reads comprise plus strand consensus sequence reads and minus strand consensus sequence reads, and wherein selecting the subset is performed using a criterion that applies to the relative numbers of plus strand consensus sequence reads and corresponding minus strand consensus sequence reads.

Example 23. The method of any one of examples 16-22, wherein determining the plurality of trinucleotide context (TNC) error rates for the respective plurality of TNC error types using the consensus sequence reads comprises:
determining the plurality of TNC error rates using background regions of the consensus sequence reads,
wherein the positions being monitored for mutations include a first position,
wherein the consensus sequence reads include a first consensus sequence read that covers the first position and the background regions include a first background region for the first consensus sequence read,
wherein the first background region comprises nucleotides in the first consensus sequence read that are at least a first threshold distance away from the first position.

Example 24. The method of example 23, wherein background regions do not include the positions being monitored for mutations.

Example 25. The method of any one of examples 16-22,
wherein the consensus sequence reads comprise, for a first position of the positions being monitored for mutations, a first group of plus strand consensus sequence reads associated with a plus strand primer binding sequence at 3′ terminal of each of the plus strand consensus sequence reads in the first group and a second group of minus strand consensus sequence reads associated with a minus strand primer binding sequence at 3′ terminal of the minus strand consensus sequence reads in the second group,
wherein determining the plurality of trinucleotide context (TNC) error rates for the respective plurality of TNC error types using the consensus sequence reads comprises: determining the plurality of TNC error rates using:
   nucleotides, in any sequence read in the first group of plus strand
      consensus sequence reads, which are located within a second threshold distance of the plus strand primer binding sequence, and
      nucleotides, in any sequence read in the second group of minus strand consensus sequence reads, which are located within a third threshold distance of the minus strand primer binding sequence.

Example 26. The method of any one of examples 16-25, wherein determining the plurality of trinucleotide context (TNC) error rates using the consensus sequence reads comprises determining a frequency of occurrence of each of the TNC error types in the consensus sequence reads.

Example 27. The method of example 20, wherein determining the plurality of trinucleotide context (TNC) error rates for the respective plurality of TNC error types using the consensus sequence reads comprises:
determining the plurality of TNC error rates from background regions of the consensus sequence reads,
wherein the consensus sequence reads include a first consensus sequence read and the background regions include a first background region for the first consensus sequence read,
wherein the TNC error rates are determined based on how often each of the TNC error types occurs in the first background region for the first consensus sequence read.

Example 28. The method of any one of examples 1-27, wherein TNC error types correspond to a mutation in any position of a given TNC.

Example 29. The method of any one of examples 1-28, wherein each of the TNC error types corresponds to a specific mutation of a middle nucleotide in a given TNC.

Example 30. The method of any one of examples 1-29, further comprising:
after determining the plurality of trinucleotide context (TNC) error rates and before grouping at least some of the plurality of TNC error rates into a plurality of TNC error rate groups, determining confidence intervals for the TNC error rates; and selecting the at least some of the plurality of TNC error rate for grouping using a criterion that applies to the confidence intervals for the TNC error rates.

Example 31. The method of any one of examples 1-30, wherein grouping at least some of the plurality of TNC error rates into a plurality of TNC error rate groups comprises clustering the plurality of TNC error rates.

Example 32. The method of any one of examples 1-31, wherein grouping at least some of the plurality of TNC error rates into a plurality of TNC error rate groups comprises grouping using partition around medoids (PAM) clustering.

Example 33. The method of any one of examples 1-32, wherein grouping at least some of the plurality of TNC error rates comprises grouping into 4 TNC error rate groups.

Example 34. The method of any one of examples 1-33, wherein determining the first value indicative of the expected number of mutations present in the sequencing data is performed using at least some of the TNC group error rates and the number of times at least some of the positions being monitored for mutations are covered by a sequence read in the first subset of sequence reads.

Example 35. The method of any one of examples 1-34, wherein determining the first value indicative of the expected number of mutations present in the sequencing data comprises:
determining the first value as a weighted linear combination of the TNC error group rates with each particular one of the TNC error group rates being weighted by a number of times a position being monitored is covered by a sequence read, in the first subset of sequence reads, corresponding to a TNC error type that belongs to that particular TNC error group.

Example 36. The method of any one of example 1-35, wherein performing (C) further comprises:
generating second consensus sequence reads using at least the second subset of the sequence reads, wherein each of the second consensus sequence reads is generated from those sequence reads, in at least the second subset of the sequence reads, which are associated with a respective common unique molecular identifier (UMI),
wherein determining the second value indicative of an actual number of mutations present at the positions being monitored for mutations is performed using the second consensus

Example 37. The method of any one of examples 1-36, wherein (D) is performed using a statistical hypothesis test having a null hypothesis, by comparing the second value to a distribution associated with the null hypothesis, wherein the distribution has one or more parameters that depend on the first value.

Example 38. The method of example 37, wherein the distribution is a Poisson distribution having a mean value (2) that is set to the first value.

Example 39. The method of example 37 or example 38, wherein using the statistical hypothesis test comprises determining a measure of likelihood, under the null hypothesis, of observing the actual number of mutations indicated by the second value.

Example 40. The method of any one of examples 1-39, wherein (D) is performed using a one-sided Poisson hypothesis test.

Example 41. The method of example 40, wherein using the one-sided Poisson hypothesis test comprises: setting a mean value (2) of a Poisson distribution to the first value and determining a measure of likelihood, under the Poisson distribution, of observing the actual number of mutations indicated by the second value.

Example 42. The method of example 41, determining whether the sequencing data provides the indication that the subject has minimum residual disease using the measure of likelihood.

Example 43. The method of any of examples 38-42, wherein the subject is likely to have minimum residual disease if the second value indicates that the null hypothesis can be rejected.

Example 44. The method of any of examples 1-43, wherein (D) further comprises: providing the indication that the subject has minimum residual disease.

Example 45. The method of any one of examples 1-44, further comprising using the at least one computer hardware processor to perform:
obtaining one or more of further sequencing data previously generated by sequencing one or more further biological sample(s) of the subject, each of the one or more of further sequencing data comprising further sequence reads covering the positions being monitored for mutations, and for each of the further sequence reads of the one or more of further sequencing data:
determining, using at least a first subset of the further sequence reads, a further first value indicative of an expected number of mutations present in a respective sequencing data due to sequencing error, the determining comprising:
   determining a further plurality of trinucleotide context (TNC) error rates for a respective plurality of TNC error types;
   grouping at least some of the further plurality of TNC error rates into a further plurality of TNC error rate groups;
   determining further TNC group error rates for the further plurality of TNC error rate groups using the further TNC error rates for the at least some of the further plurality of TNC error rates; and
   determining the further first value indicative of the expected number of mutations present in the respective sequencing data using the further TNC group error rates; determining, using at least a second subset of the further sequence reads, a further second value indicative of an actual number of mutations present at the positions; and
determining whether the respective sequencing data provides the indication that the subject has minimum residual disease using the further first value indicative of the expected number of mutations present in the respective sequencing data due to sequencing error and the further second value indicative of the actual number of mutations present in the respective sequencing data at the positions being monitored for mutations.

Example 46. A system for determining whether sequencing data of a biological sample of a subject provides an indication that the subject has minimum residual disease, the system comprising:
at least one computer hardware processor; and
at least one non-transitory computer readable storage medium storing processor executable instructions that, when executed by the at least one computer hardware processor, cause the at least one computer hardware processor to perform:
   (A) obtaining the sequencing data, the sequencing data being previously generated by sequencing the biological sample of the subject, the sequencing data comprising sequence reads covering positions being monitored for mutations;
   (B) determining, using at least a first subset of the sequence reads, a first value indicative of an expected number of mutations present in the sequencing data due to sequencing error, the determining comprising:
      determining, using the first subset of sequence reads, a plurality of trinucleotide context (TNC) error rates for a respective plurality of TNC error types;
      grouping at least some of the plurality of TNC error rates into a plurality of TNC error rate groups;
      determining TNC group error rates for the plurality of TNC error rate groups using the TNC error rates for the at least some of the plurality of TNC error rates; and
      determining the first value indicative of the expected number of mutations present in the sequencing data using the TNC group error rates;
   (C) determining, using at least a second subset of the sequence reads, a second value indicative of an actual number of mutations present at the positions being monitored for mutations; and
   (D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

Example 47. The system of example 46, wherein the at least one computer hardware processor stores processor executable instructions that cause the at least one computer hardware processor to perform the method of any of examples 2-45.

Example 48. At least one non-transitory computer readable storage medium storing processor executable instructions that, when executed by at least one computer hardware processor, cause the at least one computer hardware processor to perform:
(A) obtaining the sequencing data, the sequencing data being previously generated by sequencing the biological sample of the subject, the sequencing data comprising sequence reads covering positions being monitored for mutations;
(B) determining, using at least a first subset of the sequence reads, a first value indicative of an expected number of mutations present in the sequencing data due to sequencing error, the determining comprising:
   determining, using the first subset of sequence reads, a plurality of trinucleotide context (TNC) error rates for a respective plurality of TNC error types;
   grouping at least some of the plurality of TNC error rates into a plurality of TNC error rate groups;
   determining TNC group error rates for the plurality of TNC error rate groups using the TNC error rates for the at least some of the plurality of TNC error rates; and
   determining the first value indicative of the expected number of mutations present in the sequencing data using the TNC group error rates;
(C) determining, using at least a second subset of the sequence reads, a second value indicative of an actual number of mutations present at the positions being monitored for mutations; and
(D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

Example 49. The at least one non-transitory computer readable storage medium storing processor executable instructions of example 48, wherein the at least one computer hardware processor stores processor executable instructions that cause the at least one computer hardware processor to perform the method of any of examples 2-45.

Example 50: Determining an indication of MRD in Non-Small Cell Lung Cancer (NSCLC)

### Summary

Data is presented from a patient-specific, tumor-informed approach to ctDNA detection (e.g., an indication of MRD) that interrogated a median of 200 somatic variants per patient from the surgically excised tumor. 712 contrived samples were analyzed to assess assay performance alongside 1093 clinically annotated plasma samples from 197 prospectively recruited patients with early-stage operable NSCLC (76 of whom suffered disease recurrence). Through analyses of 383 extracranial surveillance scans it was found that MRD status could aid interpretation of equivocal findings on surveillance imaging with potential to guide early definitive intervention at these sites.

### Introduction

Circulating tumor DNA (ctDNA) is a multi-faceted biomarker with potential to accelerate innovation particularly in the early-stage interventional trial space. Post-operative ctDNA detection indicative of molecular residual disease (MRD) is a specific indicator of impending NSCLC recurrence. Here, a personalized, tumor-informed, anchored multiplex-PCR (AMP) approach to determining an indication of MRD in the evolutionary NSCLC study (NCT01888601) is reported.

MRD detection

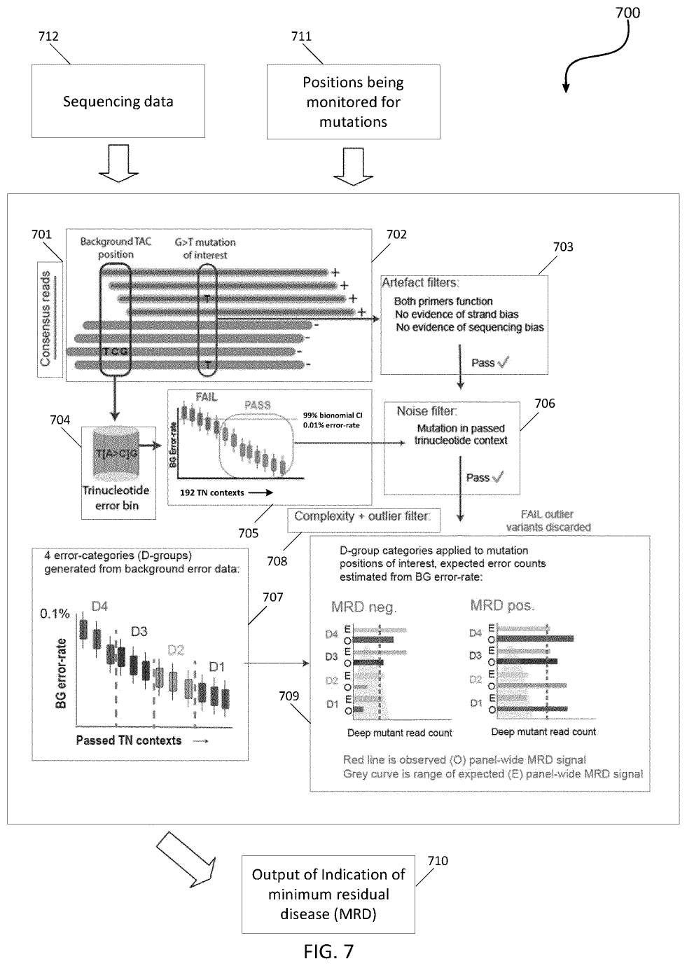

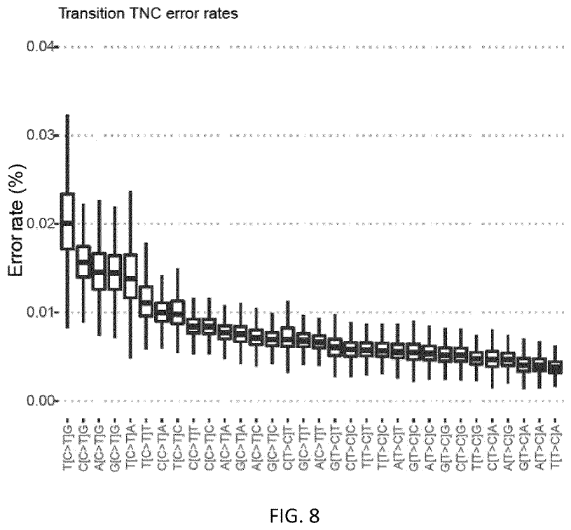

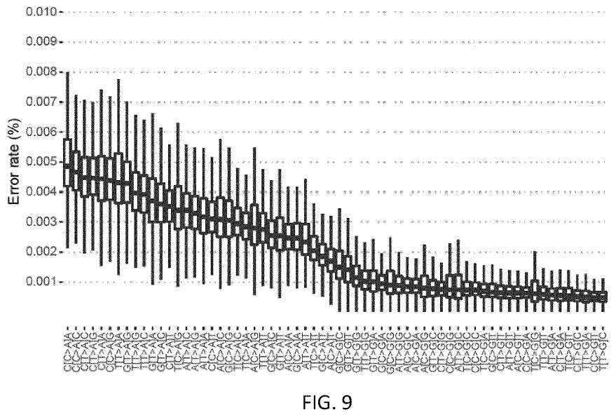

Compared to previous work, here, the number of mutations tracked by patient specific enrichment panels (e.g., PSPs or targeted gene sequencing panel) was increased, and methods to enable higher-resolution characterization of NSCLC relapse were employed (FIG. 7). PSPs targeted a median of 200 mutations (range 72 to 201). Algorithms selecting variants from tumor exome data for PSP design incorporated parameters including predicted low background sequencing error and elevated tumor copy-number status to maximize sensitivity (FIG. 8 and FIG. 9). Only deep reads were considered in cfDNA analysis to minimize sequencing error (see methods). A deep read is a read supported by a minimum number (e.g., at least 5) sequence reads associated with a single unique identifier [UMI].

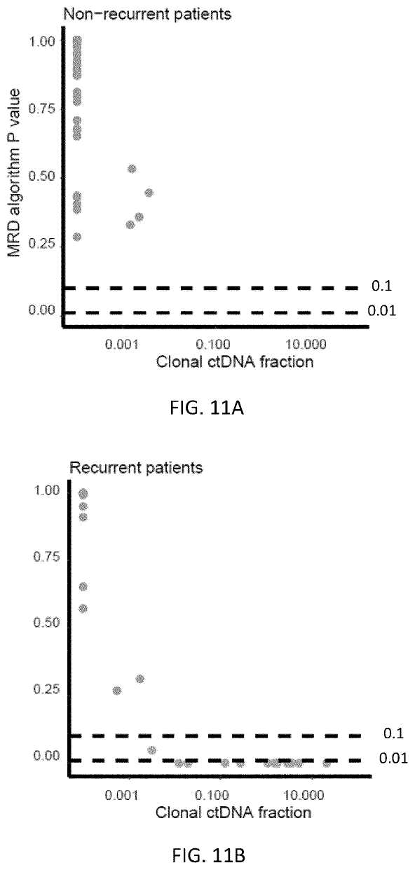

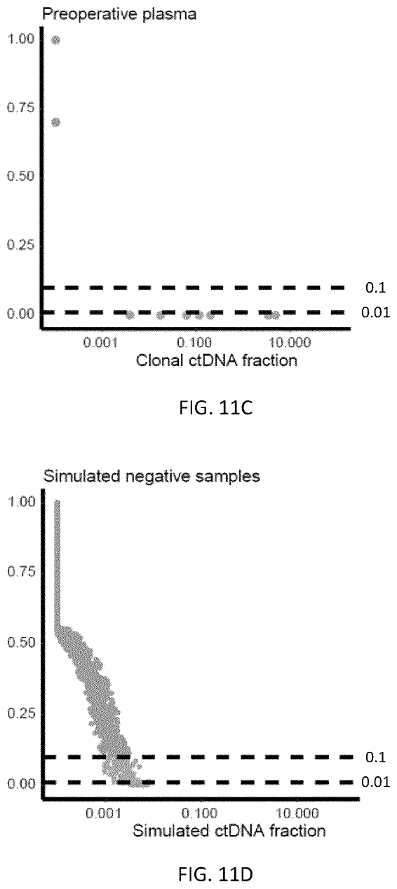

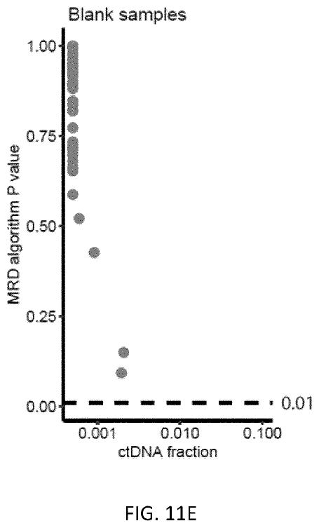

An MRD detection algorithm evaluated background (non-variant) sequencing positions to estimate intra-library tri-nucleotide context error rates, enabling ctDNA detection on a single library basis (methods, see FIG. 7). Details regarding determination of an MRD calling threshold in 10-patient pilot data, analytical validation of variant DNA detection sensitivity in 712 contrived samples analyzed with 50-variant PSPs and orthogonal validation of NSCLC pre-operative ctDNA positive calls using digital droplet PCR are presented below in “Analytical Validation Experiments” including with reference to FIG. 11E).

Post-Operative ctDNA Detection is Specific for Relapsing NSCLC

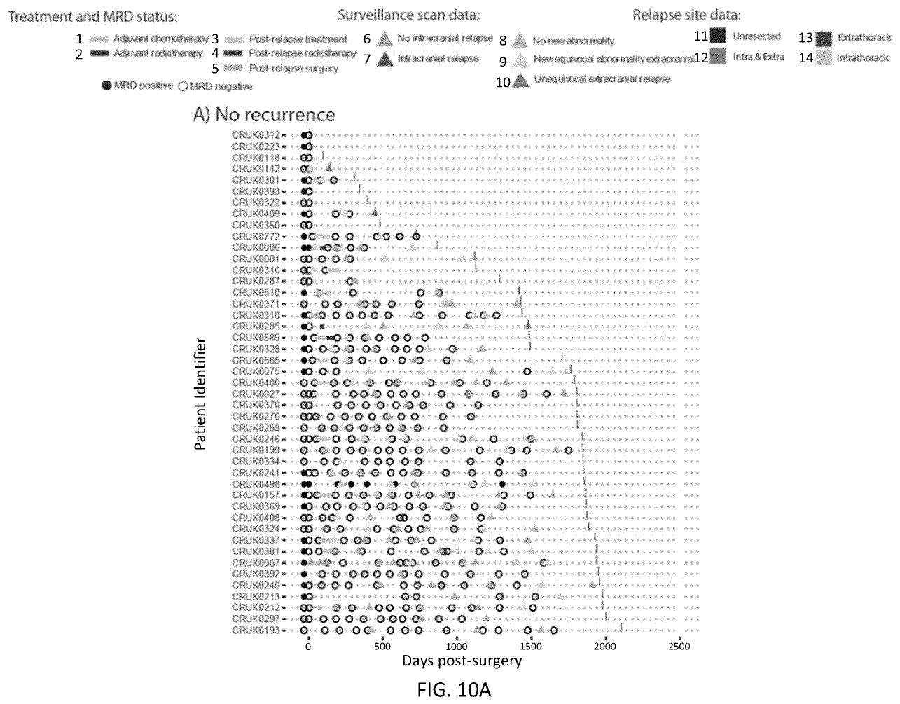

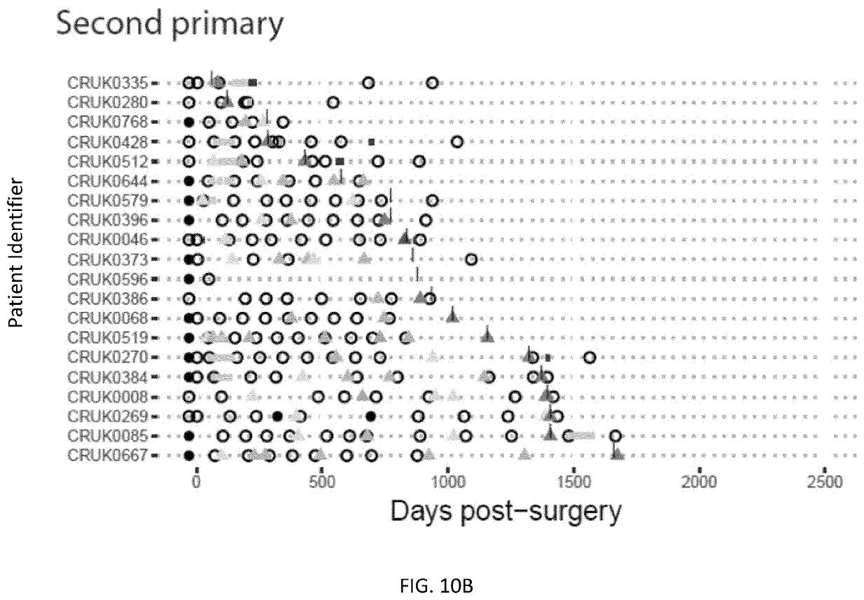

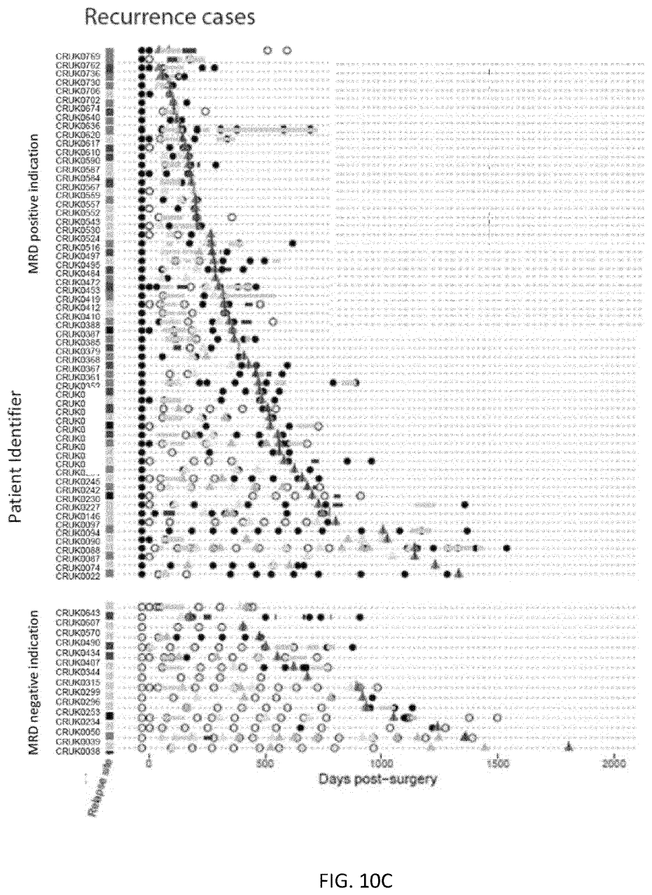

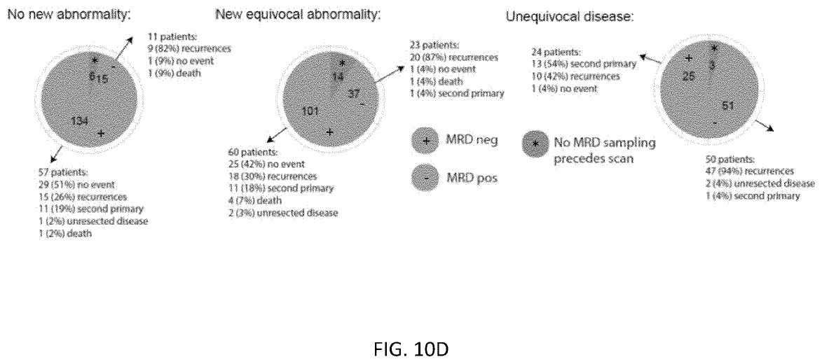

Post-operative cfDNA samples were analyzed from 45 recurrence-free patients (FIG. 10A). All samples had p-values in excess of 0.1 (FIG. 11A). Post-operative cfDNA samples were also analyzed from 20 patients who developed new second primaries during follow-up (PSPs are specific to the excised primary NSCLC and are not expected to detect second primaries, FIG. 10B). 462 of 472 (97.9%) post-operative samples from these patients were negative for ctDNA detection with 62 of 65 (95%) patients lacking post-operative ctDNA detection.

Regarding FIGS. 10A-10C, the circles to the left of day-0 are pre-operative timepoints from when the patient's tumor was still in-situ. The circles to the right of day-0 are taken following surgical excision of the primary NSCLC. If the circle is colored dark, it reflects positive ctDNA detection. The light grey rectangles (rectangles 1 and 3) represent whether a patient received chemotherapy, the dark grey rectangles (rectangles 2 and 4) represent whether a patient received radiotherapy and the medium grey shaded rectangles (rectangle 5) represent if a patient received post-recurrence surgery. The triangles represent standard of care post-operative CT, PET or MRI imaging classified as no disease (medium grey, triangle 8) equivocal images (very light grey, triangle 9) or unequivocal imaging evidence of extracranial relapse (dark grey, triangle 10). Medium grey triangles (triangle 6) represent no evidence of intracranial relapse, very dark grey triangles (triangle 7) indicate intracranial relapse. The vertical black lines represent the event date for a patient (if events such as death, second-primary, NSCLC recurrence occurred) otherwise the vertical line represents the NSCLC study (NCT01888601) follow-up censorship date for that patient.

ctDNA detection in relapsed NSCLC patients

400 postoperative plasma samples were analyzed from 76 patients who suffered recurrence of their NSCLC (FIG. 10C). In post-operative inpatients who had relapse of their NSCLC, 1 of 13 calls was made between MRD p-values of 0.1 and 0.01 (FIG. 11B). The remaining were made at a p-value less than 0.01. Within patients with detectable ctDNA prior to surgery, post-operative ctDNA was detected in 54 of 61 patients (89%). In contrast, patients lacking detectable ctDNA prior to surgery exhibited post-operative ctDNA detection in 10 of 14 cases (71%, FIG. 10C). Lead-time (days from first post-operative ctDNA detection to confirmed radiological relapse) was assessed. Patients who lacked post-operative ctDNA detection or who experienced initial ctDNA detection following clinical relapse were assigned lead-times of 0 days. 67 of 75 patients were lead-time evaluable (patients excluded with no pre-clinical recurrence plasma sampling [CRUK0048, 0557, 0516, 0674, 0640] or incompletely resected disease on post-operative imaging [CRUK0230, 0234, 0291 and 0387]). Median lead-time across all patients with detectable ctDNA prior to surgery was 119 days and mean lead-time was 236 days (range 0 to 1137 days, n=54. FIG. 10C). Median lead-time across patients without detectable ctDNA prior to surgery was 0 days and mean lead-time was 114 days (range 0 to 589 days, n=14, FIG. 10C). 26 of 52 patients were ctDNA positive at initial post-operative ctDNA sampling and 26 patients emerged ctDNA positive during surveillance after a median of 2 (range 1-9) ctDNA negative timepoints (FIG. 10C).

In reference, pre-operative ctDNA calls from pilot cohort, 7 patients had positive ctDNA in plasma prior to surgery, all calls were made at a p-value <0.01 (FIG. 11C). In-silico simulation analysis to assess ctDNA caller specificity, 3157 mock MRD panels were generated within pilot patient libraries and ctDNA caller p-values assessed (FIG. 11D). At a p-value <0.1 threshold 121/3157 simulated mock panels were ctDNA positive (in-silico specificity of 96.2%) at a P-Value <0.01 22/3157 simulated mock panels were ctDNA positive (in-silico specificity of 99.3%).

Imaging and MRD detection

386 extracranial surveillance imaging-reports from 131 patients who had postoperative plasma sampling (343 CT scans covered sites including neck, chest, abdomen, pelvis, colon, kidney, bladder, and spine, 7 Magnetic Resonance Imaging [MRI] spine, liver, or femur scans and 36 whole-body Positron Emission Tomography scans) were reviewed. MRD detection preceding scans showing no new abnormalities occurred on 15 occasions in 11 patients, 9 of whom suffered subsequent NSCLC recurrence (FIG. 10D). ctDNA detection preceding scans showing new equivocal abnormalities were common. These data suggest MRD status could guide early definitive therapeutic intervention (e.g., surgery, radiation, or ablation) at equivocal anatomic sites.

### DISCUSSION

Detection of ctDNA in the post-curative intent therapy setting indicated impending disease relapse. A total of 1096 pre- and post-operative plasma samples were analyzed from 197 patients. Superior performance of MRD surveillance was observed in patients with pre-operative ctDNA detection (median lead-time of 119 days versus 0 days in pre-operative ctDNA positive versus preoperative ctDNA negative patients) suggesting MRD surveillance could be prioritized over routine radiological surveillance in patients exhibiting pre-operative ctDNA detection. Of 12 patients with pre-adjuvant therapy MRD detection, only one remained relapse-free following adjuvant radiation therapy (CRUK0086 received post-surgical radiotherapy to the mediastinal bed and died relapse-free from bowel perforation 880 days post-surgery). As the remaining patients all experienced NSCLC recurrence up to 2.8 years following ctDNA detection. This suggests that in NSCLC, MRD detection at presented tumor-informed assay limits of detection may not identify the 8% of patients who experience 5-year survival benefit from adjuvant chemotherapy; possibly because the burden of metastatic tumor required to elicit a pre-adjuvant MRD positive result is beyond that curable with systemic therapy.

### Methods

Library preparation using Anchored-multiplex PCR| Anchored-Multiplex PCR (AMP) is a nested multiplex-PCR enrichment chemistry that incorporates strand specific priming and the incorporation of unique molecular identifiers (UMIs) into sequenced reads9. Cell-free DNA, fragmented peripheral blood mononuclear cell (PBMC) DNA or fragmented normal tissue DNA was end-repaired phosphorylated and A-tailed. An adapter containing a universal priming site, the indexes for multiplexing and a UMI is then ligated onto DNA. One round of target specific PCR was performed with a gene-specific primer 1 (GSP1) which amplifies against the P5 primer in the adapter, and a further round of PCR was then performed with a second nested gene-specific primer (GSP2) and a primer that incorporates a second primer containing a P7 index. Strand-specific priming is performed in both rounds of amplification facilitating the identification of positive and negative strand input DNA molecules during informatic analyses.

The method aimed to sequence each library to 10 million reads. The on-target deduplication ratio of the library, which describes the ratio of raw on-target reads to unique molecular identifier [UMI] supported on-target reads (UMI supported reads contained 5 or more supporting raw reads with a matched molecular index) was then evaluated. In samples where initial sequencing depth resulted in on target de-duplication ratio less than 10:1 additional sequencing was performed to maximize recovery of unique molecular index (UMI) families. PBMC and normal tissue libraries were either sequenced on the NovaSeq® 6000 system (Illumina®) or the NextSeq® system (Illumina®).

MRD Calling Algorithm | A MRD caller that investigated background sequencing noise on an intra-library basis was generated (FIG. 7). The MRD caller utilized the Archer® informatic pipeline to clean input reads and generate deduplicated UMI supported reads. The cleaned, deduplicated, and error corrected UMI-supported reads were aligned to hg19 and used to evaluate alternate observations at predefined positions where tumour-specific variants were present in the patient's tumour (tumour-informed positions). Only “deep” consensus reads supported by 5 or more PCR duplicates were used to infer expected sequencing noise as well as calculate signal for the MRD calling algorithm.

Alternate bases at tumour-informed positions were subject to a strict set of quality filters consisting of an off target filter, a read strand bias filter, a sequencing strand bias filter, background error rate filter, and variant allele frequency outlier filter to remove artefactual signals. The variant allele frequency outlier filter functioned by performing PAM (partitioning around medoids) clustering of the variant allele frequencies (VAFs) of the tumour informed positions that passed previously described filters, K was set to 2 in the clustering algorithm, thus yielding a high VAF group and a low VAF group. If one of the two clusters had significantly higher VAFs, as indicated by non-overlapping confidence intervals of the highest VAF of the low VAF cluster and the lowest VAF of the higher VAF cluster, and contained 2 or fewer tumour specific variants those variants were removed from consideration downstream in the algorithm.

Next, intra-library background error-rates (ERs) were calculated. ERs were used to establish the level of noise present in each library that had to be confidently exceeded to allow an MRD call to be made. To calculate background library ERs, the number of UMI-supported alternate observations (DAOs, deep alternate observations) were tallied across the assay's region of interest (ROI) for each trinucleotide context (TNC) and for each possible alternate position based on the plus strand of the reference sequence. The ER corresponding to each TNC alternate was calculated as DAO/DDP (DDP, deep UMI-corrected depth across a TNC alternate). In order to measure only PCR and sequencing error, a position in the ROI was not included in the TNC ER calculation if the VAF at that position for a particular alternate is >1% (on the basis this could represent a clonal haematopoiesis associated mutation or single nucleotide polymorphisms).

A mapping of tumour observed variants and their accompanied TNC ERs was generated. Any tumour observed variant with a corresponding TNC ER upper confidence interval that was above 0.01% was filtered from the MRD calling algorithm. PAM clustering was used to generate 4 “D-groups” of TNC error-rates from qualified TNCs. The population weighted average TNC error-rate was calculated for each of the four D-groups based on the product of the TNC error-rates included in each D-group cluster and the total DDP for each TNC. The generation of 4 D-groups ensured that there was sufficient intra-library DDP coverage of each D-group to make precise estimations regarding ERs for variants within each group.

To determine whether ctDNA was present in the sample, the total observed DAOs summed across tumour specific positions remaining after filters were compared to the number of DAOs that were expected due to background ERs as dictated by the D-groups. A one-tailed exact Poisson test was applied where the total remaining observed DAOs served as the value being tested and the expected number of DAOs due to error served as the lambda of the Poisson distribution. If the resulting P-value of the test is below a pre-specified alpha threshold set to 0.01 then the sample is classified as MRD positive. FIG. 11 provides data regarding how the pre-specified alpha threshold of 0.01 used in these analyses was generated.

To investigate whether a single mutation targeted by a panel was present the specific trinucleotide error-rate corresponding to the mutation of interest and a one-tail Poisson test to assess if the number of DAOs across the mutation of interest was above expected background ER were utilized. If the number of DAOs was above higher than expected background error using an alpha threshold of 0.01 then a variant was deemed confidently detected.

Designing AMP-MRD enrichment panels| tumour-informed personalized AMP-MRD enrichment panels were designed for 197 NSCLC study (NCT01888601) patients. A median of 50 variants per panel (range 0 to 50) were chosen using the ArcherDx panel design algorithm and a median of 150 variants (range 34 to 153) were chosen using variants selected from NSCLC study (NCT01888601) multi-region exome sequencing data. For Archer variant selection WES sequencing data from the highest purity tumour region and from the paired germline DNA were used. The algorithm then identified those variants for which there was high confidence that the variants were not artifacts and were tumour specific. The algorithm then determines which variants can be targeted using an ArcherDX AMP panel and from this set of variants the 50 most informative mutations are targeted based on these criteria: the quality of the primers targeting the variant (to ensure high sequencing coverage of the target variant), predicted error rate for the variant in error corrected bins and mappability. The predicted error rate for each variant is based on an analysis of AMP cfDNA libraries sequenced on a NovaSeq instrument. This error rate analysis was performed by performing targeted variant calling on every possible SNV in a set of Archer LiquidPlex cfDNA libraries. The NSCLC study variants were selected using the NSCLC primary tumour WES pipeline7 for ranking.

Each personalized enrichment panel also contained 90 primers targeting 45 common single nucleotide polymorphisms (SNPs). During analyses the zygosity of these SNPs in a cfDNA library is compared to their zygosity in the whole exome sequencing data for that patient to confirm that a sample swap did not occur. In addition the coverage provided by these primers helps in establishing the background PCR and sequencing error rate for a library. These 45 SNPs were chosen based on being present in each Gnomad subpopulation at a frequency of 25%-75% to maximize utility in detecting sample swaps.

Analytical validation experiments| For experiment LOD1, 634 samples of fragmented DNA with a known SNP profile (Genome in a Bottle DNA, NA24385) were added to a background of four other fragmented Genome in a Bottle inputs (NA24149, NA24631, NA24694 and NA24695). Six AMP enrichment panels were generated targeting 50 SNPS heterozygous in NA24385 and absent from the other four cell lines. To generate contrived samples NA24385 DNA was spiked into a background of the other four samples at ratios of 0.006% to 0.2% by mass to target variant allele frequencies ranging from 0.003% to 0.1% allele frequency (since heterozygous variants are present at 50% in the neat NA24538). As part of the same dilution series, admixtures with target allele frequencies of 1%, 5% and 10% were made. These mixtures were used as input for AMP library preparation to confirm that mixing based on mass achieved the desired target allele fractions. The spike-in variant fraction was measured in these higher AF libraries by adding the number of deep alternate reads across the targeted SNPs and dividing by the total coverage of all deep reads across targeted SNPs. This analysis confirmed that the spike-ins achieved the targeted AFs. Fragmented DNA inputs from 2 ng to 80 ng were used in the experiment to reflect the range of DNA inputs encountered in a clinical setting. Overall 564 of 634 samples were deemed evaluable for LOD1 analysis (62 samples failed because of incorrect DNA input used, determined by on-target read per primer per ng input of <30 or >400 and 8 samples failed because they had less than 10 million reads). Clinical samples were used in validation of AMP-MRD (LOD2) and were prepared using a similar method to the Genome in a Bottle mixtures. Whole exome sequencing data from four patients was used to design patient-specific panels with the ArcherDx panel design algorithm containing 50 SNVs. The panels were used to prepare libraries using cfDNA from each patient and the overall tumour variant AF for each sample was calculated by adding the total number of deep unique reads containing a targeted tumour-specific variant and dividing by the sum of the deep unique coverage across all targeted tumour variants. All four patient cfDNA libraries had a total AF of >1%. A single mixture was made using cfDNA from healthy donors and used to dilute the patient cfDNA. These dilutions were performed as a serial dilution. First a dilution was made targeting a 1% total AF and libraries were prepared using this mixture. The total AF was measured for this sample and a dilution correction factor was calculated to account for differences in conversion efficiency between the background cfDNA. E.g., if a 1% AF was targeted and an AF of 1.3% was observed then this would indicate that the patient cfDNA is more efficiently converted to library than the background and more background DNA would need to be used. Mixtures were then made to achieve AFs of 0.1%, 0.05%, 0.01%, 0.008% and 0.005%. A total of 100 libraries were prepared at 5 AFs and 3 input masses. 48 blank samples (DNA donated from 22 healthy donors) were analyzed to assess assay specificity. Panel observed allele frequencies were calculated by taking the number of deep alternate reads noted across the AMP panel, removing estimated background error and dividing by deep depth across the panel. For assay sensitivities at specific spike-in categories, Clopper-Pearson binomial two-sided 95% confidence intervals were calculated in Supplementary 2e-f using the R package DescTools and the function BinomCI.

Simulation analysis to assess specificity| Trinucleotide context of tumour-specific SNVs within each NSCLC study (NCT01888601) AMP-MRD pilot cohort panel was assessed. Based on these data mock tumour signatures (genomic positions covered by the enrichment primers with positions of similar expected error rates of the targeted SNVs) were generated. A mock variant was added to a mock signature if the following criteria was met: It is bi-directionally covered by primers intended for MRD detection, It contained the same TNC-group error rate as the true MRD variant it's replacing, it was not a known population SNP variant as dictated by Ensemble's Variant Effect Predictor version 94.5, had a error-corrected coverage delta no more than 2,000 compared with the true MRD variant, and was not used within any other mock tumour signature, including itself. Thus, the resulting mock signatures targeted bases that are not mutated in the primary tumour and any positive MRD call from these mock signatures was by default a false positive. 3157 mock signatures across 91 pilot cfDNA libraries were interrogated for MRD positive calls A simulated ctDNA fraction was estimated for each sample by taking the number of deep alternate reads noted across the mock signature, removing estimated background error and dividing by deep depth across the mock signature.

Digital droplet PCR orthogonal validation| Digital droplet polymerase chain reaction (ddPCR) orthogonal analyses were performed in 30 preoperative plasma samples from NSCLC study (NCT01888601) patients who also had preoperative plasma analyzed by the AMP-personalized tumour informed approach and 8 negative controls (preoperative plasma from patients diagnosed postoperatively with non-malignant disease). NSCLC study (NCT01888601) patients were selected as having clonal driver mutations that could be targeted by a single ddPCR assay. The ddPCR assays used were SAGAsafe® assays (SAGA diagnostics) and had been designed and developed on the BioRad QX200 Droplet Digital PCR system. ddPCR analyses were performed at SAGA, SAGA received plasma (median 4.8 mls, range 2.5 to 5.2 mls). cfDNA was extracted using the QiaAMP MinElute ccfDNA Midi Kit (Qiagen). cfDNA was eluted in 40 μ1 of Buffer EB. The entirety of cfDNA material was input in each case and ddPCR analyses were run in 4 replicate reaction wells per sample.

1. A method for determining whether sequencing data of a biological sample of a subject provides an indication that the subject has minimum residual disease, the method comprising: using at least one computer hardware processor to perform:
(A) obtaining the sequencing data, the sequencing data being previously generated by sequencing the biological sample of the subject, the sequencing data comprising sequence reads covering positions being monitored for mutations;
(B) determining, using at least a first subset of the sequence reads, a first value indicative of an expected number of mutations present in the sequencing data due to sequencing error, the determining comprising: determining, using the first subset of sequence reads, a plurality of trinucleotide context (TNC) error rates for a respective plurality of TNC error types;
grouping at least some of the plurality of TNC error rates into a plurality of TNC error rate groups; determining TNC group error rates for the plurality of TNC error rate groups using the TNC error rates for the at least some of the plurality of TNC error rates; and determining the first value indicative of the expected number of mutations present in the sequencing data using the TNC group error rates;
(C) determining, using at least a second subset of the sequence reads, a second value indicative of an actual number of mutations present at the positions being monitored for mutations; and
(D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

2. The method of claim 1, wherein the sequence reads cover at least 10 positions being monitored for mutations, or 10-200 positions being monitored for mutations, optionally wherein each of the sequence reads covers at least one of the positions being monitored for mutations.

3-4. (canceled)

5. The method of claim 1, further comprising: obtaining the sequencing data by sequencing the biological sample, optionally wherein the sequencing data comprises sequence reads from circulating tumor DNA (ctDNA).

6-9. (canceled)

10. The method of claim 1, wherein the sequence reads were obtained using a targeted gene sequencing panel, and wherein the targeted gene sequencing panel targets sequences covering positions being monitored for mutations.

11-14. (canceled)

15. The method of claim 1, wherein (B) is performed using at least the first subset of the sequence reads and one or more sequence reads in the sequencing data that do not cover the positions being monitored for mutations.

16. The method of claim 5, wherein performing (B) further comprises: generating consensus sequence reads using at least the first subset of the sequence reads, wherein each of the consensus sequence reads is generated from those sequence reads, in at least the first subset of the sequence reads, that are associated with a respective common unique molecular identifier (UMI), wherein determining the plurality of trinucleotide context (TNC) error rates for the respective plurality of TNC error types is performed using the generated consensus sequence reads, optionally wherein each of the consensus sequence reads is generated from at least a threshold number of sequence reads that are associated with a respective common UMI, and optionally wherein the threshold number of sequence reads is between 2 and 20.

17-18. (canceled)

19. The method of claim 16, further comprising: selecting a subset of the consensus sequence reads, wherein determining the plurality of trinucleotide context (TNC) error rates for the respective plurality of TNC error types is performed using only the selected subset of consensus sequence reads, optionally wherein the consensus sequence reads comprise plus strand consensus sequence reads and minus strand consensus sequence reads, and wherein selecting the subset is performed using a criterion that applies a measure of similarity between corresponding plus strand consensus sequence reads and minus strand consensus sequence reads and optionally wherein the consensus sequence reads comprise plus strand consensus sequence reads and minus strand consensus sequence reads, and selecting a subset of the consensus reads using one or more criteria that apply to the plus strand consensus sequence reads and minus strand consensus sequence reads.

20-22. (canceled)

23. The method of claim 5, wherein determining the plurality of trinucleotide context (TNC) error rates for the respective plurality of TNC error types using the consensus sequence reads comprises: determining the plurality of TNC error rates using background regions of the consensus sequence reads, wherein the positions being monitored for mutations include a first position, wherein the consensus sequence reads include a first consensus sequence read that covers the first position and the background regions include a first background region for the first consensus sequence read, wherein the first background region comprises nucleotides in the first consensus sequence read that are at least a first threshold distance away from the first position.

24. (canceled)

25. The method of claim 5, wherein the consensus sequence reads comprise, for a first position of the positions being monitored for mutations, a first group of plus strand consensus sequence reads associated with a plus strand primer binding sequence at the 3 ‘terminal of each of the plus strand consensus sequence reads in the first group and a second group of minus strand consensus sequence reads associated with a minus strand primer binding sequence at the 3’ terminal of the minus strand consensus sequence reads in the second group, wherein determining the plurality of trinucleotide context (TNC) error rates for the respective plurality of TNC error types using the consensus sequence reads comprises: determining the plurality of TNC error rates using: nucleotides, in any sequence read in the first group of plus strand consensus sequence reads, which are located within a second threshold distance of the plus strand primer binding sequence, and nucleotides, in any sequence read in the second group of minus strand consensus sequence reads, which are located within a third threshold distance of the minus strand primer binding sequence.

26. The method of claim 5, wherein determining the plurality of trinucleotide context (TNC) error rates using the consensus sequence reads comprises determining a frequency of occurrence of each of the TNC error types in the consensus sequence reads, or optionally wherein determining the plurality of trinucleotide context (TNC) error rates for the respective plurality of TNC error types using the consensus sequence reads comprises: determining the plurality of TNC error rates from background regions of the consensus sequence reads, wherein the consensus sequence reads include a first consensus sequence read and the background regions include a first background region for the first consensus sequence read, wherein the TNC error rates are determined based on how often each of the TNC error types occurs in the first background region for the first consensus sequence read.

27. (canceled)

28. The method of claim 1, wherein TNC error types correspond to a mutation in any position of a given TNC, or wherein each of the TNC error types corresponds to a specific mutation of a middle nucleotide in a given TNC.

29. (canceled)

30. The method of claim 1, further comprising: after determining the plurality of trinucleotide context (TNC) error rates and before grouping at least some of the plurality of TNC error rates into a plurality of TNC error rate groups, determining confidence intervals for the TNC error rates; and selecting the at least some of the plurality of TNC error rate for grouping using a criterion that applies to the confidence intervals for the TNC error rates optionally a) wherein grouping at least some of the plurality of TNC error rates into a plurality of TNC error rate groups comprises clustering the plurality of TNC error rates, b) wherein grouping at least some of the plurality of TNC error rates into a plurality of TNC error rate groups comprises grouping using partition around medoids (PAM) clustering, and/or c) wherein grouping at least some of the plurality of TNC error rates comprising grouping into 4 TNC error rate groups.

31-33. (canceled)

34. The method of claim 1, wherein determining the first value indicative of the expected number of mutations present in the sequencing data is performed using at least some of the TNC group error rates and the number of times at least some of the positions being monitored for mutations are covered by a sequence read in the first subset of sequence reads, and/or wherein determining the first value indicative of the expected number of mutations present in the sequencing data comprises: determining the first value as a weighted linear combination of the TNC error group rates with each particular one of the TNC error group rates being weighted by a number of times a position being monitored is covered by a sequence read, in the first subset of sequence reads, corresponding to a TNC error type that belongs to that particular TNC error group.

35. (canceled)

36. The method of claim 1, wherein performing (C) further comprises: generating second consensus sequence reads using at least the second subset of the sequence reads, wherein each of the second consensus sequence reads is generated from those sequence reads, in at least the second subset of the sequence reads, which are associated with a respective common unique molecular identifier (UMI), wherein determining the second value indicative of an actual number of mutations present at the positions being monitored for mutations is performed using the second consensus sequence reads.

37. The method of claim 1, wherein (D) is performed using a statistical hypothesis test having a null hypothesis, by comparing the second value to a distribution associated with the null hypothesis, wherein the distribution has one or more parameters that depend on the first value, optionally wherein the distribution is a Poisson distribution having a mean value (X) that is set to the first value, optionally wherein using the statistical hypothesis test comprises determining a measure of likelihood, under the null hypothesis, of observing the actual number of mutations indicated by the second value, and optionally wherein (D) is performed using a one-sided Poisson hypothesis test.

38-40. (canceled)

41. The method of claim 37, wherein using the one-sided Poisson hypothesis test comprises: setting a mean value (X) of a Poisson distribution to the first value and determining a measure of likelihood, under the Poisson distribution, of observing the actual number of mutations indicated by the second value, optionally wherein determining whether the sequencing data provides the indication that the subject has minimum residual disease using the measure of likelihood, and/or wherein the subject is likely to have minimum residual disease if the second value indicates that the null hypothesis can be rejected.

42-43. (canceled)

44. The method of claim 1, wherein (D) further comprises: providing the indication that the subject has minimum residual disease.

45. The method of claim 1, further comprising using the at least one computer hardware processor to perform: obtaining one or more of further sequencing data previously generated by sequencing one or more further biological sample(s) of the subject, each of the one or more of further sequencing data comprising further sequence reads covering the positions being monitored for mutations, and for each of the further sequence reads of the one or more of further sequencing data: determining, using at least a first subset of the further sequence reads, a further first value indicative of an expected number of mutations present in a respective sequencing data due to sequencing error, the determining comprising: determining a further plurality of trinucleotide context (TNC) error rates for a respective plurality of TNC error types; grouping at least some of the further plurality of TNC error rates into a further plurality of TNC error rate groups; determining further TNC group error rates for the further plurality of TNC error rate groups using the further TNC error rates for the at least some of the further plurality of TNC error rates; and determining the further first value indicative of the expected number of mutations present in the respective sequencing data using the further TNC group error rates; determining, using at least a second subset of the further sequence reads, a further second value indicative of an actual number of mutations present at the positions; and determining whether the respective sequencing data provides the indication that the subject has minimum residual disease using the further first value indicative of the expected number of mutations present in the respective sequencing data due to sequencing error and the further second value indicative of the actual number of mutations present in the respective sequencing data at the positions being monitored for mutations.

46. A system for determining whether sequencing data of a biological sample of a subject provides an indication that the subject has minimum residual disease, the system comprising: at least one computer hardware processor; and at least one non-transitory computer readable storage medium storing processor executable instructions that, when executed by the at least one computer hardware processor, cause the at least one computer hardware processor to perform:
(A) obtaining the sequencing data, the sequencing data being previously generated by sequencing the biological sample of the subject, the sequencing data comprising sequence reads covering positions being monitored for mutations;
(B) determining, using at least a first subset of the sequence reads, a first value indicative of an expected number of mutations present in the sequencing data due to sequencing error, the determining comprising: determining, using the first subset of sequence reads, a plurality of trinucleotide context (TNC) error rates for a respective plurality of TNC error types;
grouping at least some of the plurality of TNC error rates into a plurality of TNC error rate groups; determining TNC group error rates for the plurality of TNC error rate groups using the TNC error rates for the at least some of the plurality of TNC error rates; and determining the first value indicative of the expected number of mutations present in the sequencing data using the TNC group error rates;
(C) determining, using at least a second subset of the sequence reads, a second value indicative of an actual number of mutations present at the positions being monitored for mutations; and
(D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

47. (canceled)

48. At least one non-transitory computer readable storage medium storing processor executable instructions that, when executed by at least one computer hardware processor, cause the at least one computer hardware processor to perform:
(A) obtaining the sequencing data, the sequencing data being previously generated by sequencing the biological sample of the subject, the sequencing data comprising sequence reads covering positions being monitored for mutations;
(B) determining, using at least a first subset of the sequence reads, a first value indicative of an expected number of mutations present in the sequencing data due to sequencing error, the determining comprising: determining, using the first subset of sequence reads, a plurality of trinucleotide context (TNC) error rates for a respective plurality of TNC error types;
grouping at least some of the plurality of TNC error rates into a plurality of TNC error rate groups; determining TNC group error rates for the plurality of TNC error rate groups using the TNC error rates for the at least some of the plurality of TNC error rates; and determining the first value indicative of the expected number of mutations present in the sequencing data using the TNC group error rates;
(C) determining, using at least a second subset of the sequence reads, a second value indicative of an actual number of mutations present at the positions being monitored for mutations; and
(D) determining whether the sequencing data provides the indication that the subject has minimum residual disease using the first value indicative of the expected number of mutations present in the sequencing data due to sequencing error and the second value indicative of the actual number of mutations present in the sequencing data at the positions being monitored for mutations.

49. (canceled)
